(19) United States
(12) Patent Application Publication
(10) Pub. No.: US 2010/0009949 A1
(43) Pub. Date: Jan. 14, 2010

(54) NOVEL METHOD

(75) Inventor: Luciano Adorini, Milan (IT)

Correspondence Address: EDWARDS ANGELL PALMER & DODGE LLP, P.O. BOX 55874, BOSTON, MA, 02205, US

(73) Assignee: BioXell S.p.A., Milan (IT)

(21) Appl. No.: 12/280,393

(22) PCT Filed: Mar. 24, 2001
(86) PCT No.: PCT/EP06/61044

§ 371 Date: Dec. 9, 2008

(51) Int. Cl. A61K 31/59; A61P 35/00

(52) U.S. Cl. 514/167

(57) ABSTRACT

The invention provides for the use of Vitamin D compounds such as 1-alpha-fluoro-25-hydroxy-16,23E-diene-26,27-bishomo-20-epi-chole-calciferol, in the prevention or treatment of prostate cancer.

### BACKGROUND OF THE INVENTION

The importance of vitamin D (cholecalciferol) in the biological systems of higher animals has been recognized since its discovery by Mellanby in 1920 (Mellanby, E. (1921) Spec. Rep. Ser. Med. Res. Council (GB) SRS 61:4). It was in the interval of 1920-1930 that vitamin D officially became classified as a “vitamin” that was essential for the normal development of the skeleton and maintenance of calcium and phosphorous homeostasis.

Studies involving the metabolism of vitamin D₃ were initiated with the discovery and chemical characterization of the plasma metabolite, 25-hydroxyvitamin D₃ [25(OH)D₃] (Blunt, J. W. et al. (1968) Biochemistry 6:3317-3322) and the hormonally active form, 1-alpha,25(OH)₂D₃ (Myrtle, J. F. et al. (1970) J. Biol. Chem. 245:1190-1196; Norman, A. W. et al. (1971) Science 173:51-54; Lawson, D. E. M. et al. (1971) Nature 230:228-230; Holick, M. F. (1971) Proc. Natl. Acad. Sci. USA 68:803-804). The formulation of the concept of a vitamin D endocrine system was dependent both upon appreciation of the key role of the kidney in producing 1-alpha, 25(OH)₂D₃ in a carefully regulated fashion (Fraser, D. R. and Kodicek, E (1970) Nature 288:764-766; Wong, R. G. et al. (1972) J. Clin. Invest. 51:1287-1291), and the discovery of a nuclear receptor for 1-alpha,25(OH)₂D₃ (VD₃R) in the intestine (Haussler, M. R. et al. (1969) Exp. Cell Res. 58:234-242; Tsai, H. C. and Norman, A. W. (1972) J. Biol. Chem. 248:5967-5975).

The operation of the vitamin D endocrine system depends on the following: first, on the presence of cytochrome P450 enzymes in the liver (Bergman, T. and Postlind, H. (1991) Biochem. J. 276:427-432; Ohyama, Y. and Okuda, K. (1991) J. Biol. Chem. 266:8690-8695) and kidney (Henry, H. L. and Norman, A. W. (1974) J. Biol. Chem. 249:7529-7535; Gray, R. W. and Ghazarian, J. G. (1989) Biochem. J. 259:561-568), and in a variety of other tissues to effect the conversion of vitamin D₃ into biologically active metabolites such as 1alpha,25(OH)₂D₃ and 24R,25(OH)₂D₃; second, on the existence of the plasma vitamin D binding protein (DBP) to effect the selective transport and delivery of these hydrophobic molecules to the various tissue components of the vitamin D endocrine system (Van Baelen, H. et al. (1988) Ann NY Acad. Sci. 538:60-68; Cooke, N.E. and Haddad, J. G. (1989) Endocr. Rev. 10:294-307; Bikle, D. D. et al. (1986) J. Clin. Endocrinol. Metab. 63:954-959); and third, upon the existence of stereoselective receptors in a wide variety of target tissues that interact with the agonist 1alpha,25(OH)₂D₃ to generate the requisite specific biological responses for this secosteroid hormone (Pike, J. W. (1991) Annu. Rev. Nutr. 11: 189-216). To date, there is evidence that nuclear receptors for 1-alpha, 25(OH)₂D₃ (VD₃R) exist in more than 30 tissues and cancer cell lines (Reichel, H. and Norman, A. W. (1989) Annu. Rev. Med. 40:71-78).

Given the activities of vitamin D₃ and its metabolites, much attention has focused on the development of synthetic analogs of these compounds. A large number of these analogs involve structural modifications in the A ring, B ring, C/D rings, and, primarily, the side chain (Bouillon, R. et al., Endocrine Reviews 16(2):201-204). Although a vast majority of the vitamin D₃ analogs developed to date involve structural modifications in the side chain, a few studies have reported the biological profile of A-ring diastereomers (Norman, A. W. et al. (1993) J. Biol. Chem. 268 (27): 20022-20030). Furthermore, biological esterification of steroids has been studied (Hochberg, R. B., (1998) Endocr Rev. 19(3): 331-348), and esters of vitamin D₃ are known (WO 97/11053).

Prostate Cancer (PC) is one of the most common cancers and is the second leading cause of death in American men (Gronberg H. (2003) Lancet 361:859-864). In the advanced stages of the disease androgen ablation therapy represents a valuable tool for the treatment of these patients. However, in almost all patients androgen-independent (AI) clones of tumor cells develop after a year of treatment and at this stage no other efficacious therapies are available. The mechanisms responsible for transition to androgen-independence are still unclear (So et al. 2005J. Urol. 23:1-9), however, a striking characteristic of AI-PC is related to its higher invasive potential compared to androgen-dependent stages (Chung et al. (2005) J. Urol. 173:10-20). In vitro studies using available androgen-sensitive and -insensitive human PC cell lines indicate that, at least in part, higher invasiveness of AI-PC may be due to loss of regulation of genes involved in invasion (Baldi et al. 2003 Endocrinology 144:1653-1655). Bone metastases have been reported to occur in 85 to 100% of patients with advanced PC. Thus novel therapies aiming to increase the survival chance and the quality of life of patients with advanced PC should focus on inhibiting the invasive potential of the tumor as well as its proliferation.

Based on their anti-proliferative, pro-apoptotic and pro-differentiative properties, vitamin D analogs have been extensively studied as possible treatments for cancer (Nagpal et al, 2005 Endocr. Rev. 26:662-87).

Several studies have focused on the role of calcitriol and its receptor, the vitamin D receptor (VDR), in PC (Pheel and Feldman 2004 J. Steroid Biochem. Mol. Biol. 92:307-315) and clinical trials have shown the capacity of calcitriol to inhibit PSA increase in PC patients (Trump et al, 2004 J. Steroid Biochem. Mol. Biol. 89-90:519-26). Polymorphisms in the VDR gene have been implicated as risk factors for PC development and progression (Habuchi et al. 2000 Cancer Res. 60:305-308) and the growth inhibitory effects of calcitriol and its analogues have been well characterized in PC cells (Nagpal et al. 2005 Endocr. Rev. 26:662-87). However, much less is known about the effect of these compounds on the invasive ability of PC cells, although calcitriol has been shown to reduce invasion in PC cells (Sung and Feldman. 2000 Mol. Cell. Endocrinol. 164:133-143; Schwartz et al. 1997 Cancer Epidemiol. Biomarkers Prev. 6:727-732).

A major problem with the clinical use of calcitriol is its hypercalcemia-inducing capacity, prompting the search for less hypercalcemic analogues. Some vitamin D analogs are less hypercalcemic and show a strong antiproliferative activity in PC cell lines and benign stromal cells in vitro, being effective at very low concentrations (Crescioli et al. 2000 J. Clin. Endocrinol. Metab. 85:2576-2583 and Crescioli et al. 2002 Prostate 50:15-26). 1,25-dihydroxy-16ene-23yne vitamin D₃ inhibits in vitro growth of both BPH and PC cells by disrupting KGF-induced growth, decreasing bcl-2 over-expression and inducing apoptosis (Crescioli et al. 2002 Prostate 50:15-26 and 2003 Endocrinology 144:3046-3057). Strikingly, the effect of the compound on KGF-induced growth is mediated by inhibition of KGF-induced KGF receptor (KGFR) autotransphosphorylation following a brief (5 minutes) treatment (Crescioli et al. 2002 Prostate 50:15-26), indicating the involvement of a rapid, nongenomic mechanism of the vitamin D analogue on growth inhibition in PC.

The invention is based upon findings from an investigation of the effect of 1-alpha-fluoro-25-hydroxy-16,23E-diene-26,27-bishomo-20-epi-cholecalciferol on KGF-induced invasion and proliferation of the androgen-independent PC cell line DU145. Previous data from the Inventors demonstrated the capacity of this analogue to decrease prostate cell proliferation both in vitro, using primary cultures of human BPH cells and in vivo, showing inhibition of prostate growth in intact and castrated, testosterone-replaced, rats (Crescioli et al. 2004 Eur. J. Endocrinol. 150:591-603). Based on these data, 1-alpha-fluoro-25-hydroxy-16,23E-diene-26,27-bishomo-20-epi-cholecalciferol is currently being tested in phase II trials for the treatment of benign prostate hyperplasia.

Therefore a strong need exists for more selective and specific treatment of prostate cancer which is free of the well recognised disadvantages of the current treatments.

### SUMMARY OF THE INVENTION

The present invention provides for the use of vitamin D compounds, in particular vitamin D compounds of formula (I) and especially 1-alpha-fluoro-25-hydroxy-16,23E-diene-26,27-bishomo-20-epi-cholecalciferol, for the prevention or treatment of prostate cancer (PC) and associated symptoms. It further provides a method for preventing or treating prostate cancer and associated symptoms by administering a vitamin D compound, in particular vitamin D compounds of formula (I) and especially 1-alpha-fluoro-25-hydroxy-16,23E-diene-26,27-bishomo-20-epi-cholecalciferol, in an amount effective to prevent or to treat such disease alone or in combination with further active agents.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

Thus, in one aspect, the invention provides a method for preventing or treating prostate cancer in a subject, comprising administering to a subject in need thereof a therapeutically effective amount of a vitamin D compound of formula (I):

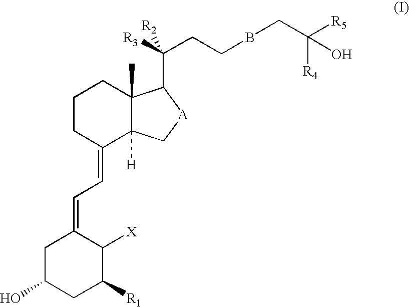

wherein:

### X is H₂ or CH₂;

R₁ is hydrogen, hydroxy or fluoro;
R₂ is hydrogen or methyl;
R₃ is hydrogen or methyl, wherein both R₂ and R₃ cannot both be hydrogen;
R₄ is methyl, ethyl or trifluoromethyl;
R₅ is methyl, ethyl or trifluoromethyl;
A is a single or double bond; and
B is a single, E-double, Z-double or triple bond; and pharmaceutically acceptable esters, salts, and prodrugs thereof; such that prostate cancer is prevented or treated in the subject.

In a preferred embodiment, the vitamin D compound is 1-alpha-fluoro-25-hydroxy-16,23E-diene-26,27-bishomo-20-epi-cholecalciferol (Compound 1):

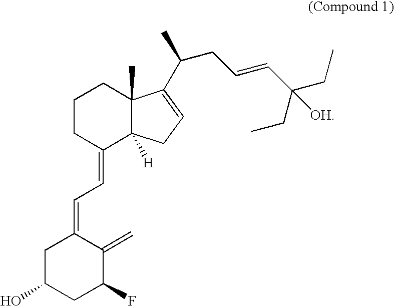

In another aspect, the invention provides a pharmaceutical composition comprising vitamin D compound, such as a vitamin D compound of formula (I), and a pharmaceutically acceptable carrier.

In yet another aspect, the invention provides a kit comprising a vitamin D compound, such as a vitamin D compound of formula (I), packaged together with instructions directing administration of said compound to a subject in need of treatment or prevention of prostate cancer in accordance with the methods of the invention.

In a further aspect there is provided a vitamin D compound for use in the treatment or prevention of prostate cancer.

Also provided is the use of a vitamin D compound in the manufacture of a medicament for the treatment or prevention of prostate cancer.

Also provided is a vitamin D compound for use in the treatment or prevention of prostate cancer.

There is additionally provided a pharmaceutical combination comprising a vitamin D compound and a further agent (such as an alpha adrenergic receptor blocking agent or a 5-alpha reductase inhibitor) for the treatment or prevention of prostate cancer.

Suitably the methods and uses of the invention are directed towards the prevention or treatment of prostate cancer without anti-androgenic prostatic and extra-prostatic adverse effects.

The methods and uses of the invention are expected to be of particular interest in the prevention or treatment of androgen independent prostate cancer (i.e. advanced prostate cancer).

### BRIEF DESCRIPTION OF THE DRAWINGS

FIG. 1: Effect of 1-alpha-fluoro-25-hydroxy-16,23E-diene-26,27-bishomo-20-epi-cholecalciferol on basal (inset) and KGF-stimulated proliferation of DU145 cells, determined by cell counting. Cells were treated for 48 hours with increased concentrations of the analogue with or without fixed concentrations of KGF (10 ng/ml). Each experimental point was determined in triplicate and experiments were performed at least three times. Results are expressed as the percentage of growth compared with their relative controls.

FIG. 2: Effect of 1-alpha-fluoro-25-hydroxy-16,23E-diene-26,27-bishomo-20-epi-cholecalciferol (1×10⁻⁸ M) on Matrigel invasion of DU145 cells (A) and PC3 cells (B) in basal conditions and following stimulation with KGF (10 ng/ml). Matrigel invasion was evaluated by using Boyden chambers. Number of cells migrated was evaluated in at least 10 fields for each experimental point and averaged. Data are means±SEM of the percentage of cell migrated respect to control of 4 different experiments.

FIG. 3: Effect of 1-alpha-fluoro-25-hydroxy-16,23E-diene-26,27-bishomo-20-epi-cholecalciferol (1×10⁻⁸ M) on KGF (10 ng/ml)-induced autotransphosphorylation of their respective receptors. Cells were pre-treated (B) or not (A) for 4 hours with alpha-amanitin (4 ug/ml, 4 h). After stimulations, cell lysates were immunoprecipitated using anti-KGFR antibody, run onto SDS-PAGE and analyzed first for expression of phosphorylation using anti-phosphotyrosine (PY20) antibody (upper blots) and, after stripping and re-probing, for receptor expression using anti-KGFR and, for the experiment with alpha-amanitin, for actin expression.

FIG. 4: Effect of the phosphatidylinositol-3kinase inhibitor, LY294002, on proliferation of DU145 cells, determined by cell counting. Cells were treated for 48 hours with fixed concentration of LY294002 (10 nM) with or without KGF (10 ng/ml). Each experimental point was determined in triplicate and experiments were performed at least three times. Results are expressed as the percentage of growth compared with their relative controls.

FIG. 5: Effect of 1-alpha-fluoro-25-hydroxy-16,23E-diene-26,27-bishomo-20-epi-cholecalciferol (1×10⁻⁸ M) on KGF (10 ng/ml)-mediated PI3K activation. After stimulation, cell lysates were immunoprecipitated using an anti-phosphotyrosine (PY20) antibody, followed by immunokinase assay in the presence of [gamma-³²P]ATP (for details, see materials and methods). Products of the reaction are evaluated by thin-layer chromatography followed by autoradiography. Upper panels show a representative experiments, where spots correspond to the PI3-kinase catalytic product [³²P]phosphatidylinositol phosphate (PIP), while lower panels show mean±SEM quantification (arbitrary Units) of the band for the indicated number of experiments.

FIG. 6: Effect of 1-alpha-fluoro-25-hydroxy-16,23E-diene-26,27-bishomo-20-epi-cholecalciferol (1×10⁻⁸ M) on KGF (10 ng/ml)-mediated phosphorylation of the PI3K downstream effector AKT. After stimulation, equal amount of total cell lysates were subjected to SDS-PAGE, transferred to nitrocellulose membranes and blotted with anti-phosphoserine AKT antibodies (upper panels) followed by stripping and re-probing with anti-AKT antibodies (lower panels). Representative of 2 similar experiments.

### DETAILED DESCRIPTION OF THE INVENTION

### Definitions

Before a further description of the present invention, and in order that the invention may be more readily understood, certain terms are first defined and collected here for convenience.

The term “administration” or “administering” includes routes of introducing the vitamin D compound(s) to a subject to perform their intended function. Examples of routes of administration which can be used include injection (subcutaneous, intravenous, parenterally, intraperitoneally, oral, inhalation, rectal, transdermal or via bladder instillation. The pharmaceutical preparations are, of course, given by forms suitable for each administration route. For example, these preparations are administered in tablets or capsule form, by injection, infusion, inhalation, lotion, ointment, suppository, etc. Oral administration is preferred. The injection can be bolus or can be continuous infusion. Depending on the route of administration, the vitamin D compound can be coated with or disposed in a selected material to protect it from natural conditions which may detrimentally effect its ability to perform its intended function. The vitamin D compound can be administered alone, or in conjunction with either another agent useful in the treatment of prostate cancer, or with a pharmaceutically-acceptable carrier, or both. The vitamin D compound can be administered prior to the administration of the other agent, simultaneously with the agent, or after the administration of the agent. Furthermore, the vitamin D compound can also be administered in a pro-form which is converted into its active metabolite, or more active metabolite in vivo.

The term “effective amount” includes an amount effective, at dosages and for periods of time necessary, to achieve the desired result, i.e. sufficient to treat prostate cancer. An effective amount of vitamin D compound may vary according to factors such as the disease state, age and weight of the subject, and the ability of the vitamin D compound to elicit a desired response in the subject. Dosage regimens may be adjusted to provide the optimum therapeutic response. An effective amount is also one in which any toxic or detrimental effects (e.g., side effects) of the vitamin D compound are outweighed by the therapeutically beneficial effects.

A therapeutically effective amount of vitamin D compound (i.e., an effective dosage) may range from about 0.001 to 30 ug/kg body weight, preferably about 0.01 to 25 ug/kg body weight, more preferably about 0.1 to 20 ug/kg body weight, and even more preferably about 1 to 10 ug/kg, 2 to 9 ug/kg, 3 to 8 ug/kg, 4 to 7 ug/kg, or 5 to 6 ug/kg body weight. The skilled artisan will appreciate that certain factors may influence the dosage required to effectively treat a subject, including but not limited to the severity of the disease or disorder, previous treatments, the general health and/or age of the subject, and other diseases present. In addition, the dose administered will also depend on the particular vitamin D compound used, the effective amount of each compounds can be determined by titration methods known in the art. Moreover, treatment of a subject with a therapeutically effective amount of a vitamin D compound can include a single treatment or, preferably, can include a series of treatments. In one example, a subject is treated with a vitamin D compound in the range of between about 0.1 to 20 ug/kg body weight, one time per day for a duration of six months or longer, for example for life depending on management of the symptoms and the evolution of the condition.

Also, as with other chronic treatments an “on-off” or intermittent treatment regime can be considered. It will also be appreciated that the effective dosage of a vitamin D compound used for treatment may increase or decrease over the course of a particular treatment.

The term “alkyl” refers to the radical of saturated aliphatic groups, including straight-chain alkyl groups, branched-chain alkyl groups, cycloalkyl(alicyclic) groups, alkyl substituted cycloalkyl groups, and cycloalkyl substituted alkyl groups. The term alkyl further includes alkyl groups, which can optionally further include (for example, in one embodiment alkyl groups do not include) oxygen, nitrogen, sulfur or phosphorus atoms replacing one or more carbons of the hydrocarbon backbone, e.g., oxygen, nitrogen, sulfur or phosphorus atoms. In preferred embodiments, a straight chain or branched chain alkyl has 30 or fewer carbon atoms in its backbone (e.g., C₁-C₃₀ for straight chain, C₃-C₃₀ for branched chain), preferably 26 or fewer, and more preferably 20 or fewer, especially 6 or fewer. Likewise, preferred cycloalkyls have from 3-10 carbon atoms in their ring structure, and more preferably have 3, 4, 5, 6 or 7 carbons in the ring structure.

Moreover, the term alkyl as used throughout the specification and claims is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above.

An “alkylaryl” moiety is an alkyl substituted with an aryl (e.g., phenylmethyl(benzyl)). Unsubstituted alkyl (including cycloalkyl) groups or groups substituted by halogen, especially fluorine, are generally preferred over other substituted groups. The term “alkyl” also includes unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six, and most preferably from one to four carbon atoms in its backbone structure, which may be straight or branched-chain. Examples of lower alkyl groups include methyl, ethyl, propyl(n-propyl and i-propyl), butyl(tert-butyl, n-butyl and sec-butyl), pentyl, hexyl, heptyl, octyl and so forth. In preferred embodiment, the term “lower alkyl” includes a straight chain alkyl having 4 or fewer carbon atoms in its backbone, e.g., C₁-C₄ alkyl.

Thus specific examples of alkyl include C₁₋₆ alkyl or C₁₋₄alkyl (such as methyl or ethyl). Specific examples of hydroxyalkyl include C₁₋₆hydroxyalkyl or C₁₋₄hydroalkyl (such as hydroxymethyl).

The terms “alkoxyalkyl,” “polyaminoalkyl” and “thioalkoxyalkyl” refer to alkyl groups, as described above, which further include oxygen, nitrogen or sulfur atoms replacing one or more carbons of the hydrocarbon backbone, e.g., oxygen, nitrogen or sulfur atoms.

The term “aryl” as used herein, refers to the radical of aryl groups, including 5- and 6-membered single-ring aromatic groups that may include from zero to four heteroatoms, for example, benzene, pyrrole, furan, thiophene, imidazole, benzoxazole, benzothiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like.

Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The terms “alkenyl” and “alkynyl” refer to unsaturated aliphatic groups analoguous in length and possible substitution to the alkyls described above, but that contain at least one double or triple bond, respectively. For example, the invention contemplates cyano and propargyl groups.

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another.

The term “enantiomers” refers to two stereoisomers of a compound which are non-superimposable mirror images of one another. An equimolar mixture of two enantiomers is called a “racemic mixture” or a “racemate.”

As used herein, the term “halogen” designates —F, —Cl, —Br or —I; the term “sulfhydryl” or “thiol” means —SH; the term “hydroxyl” means —OH.

The term “haloalkyl” is intended to include alkyl groups as defined above that are mono-, di- or polysubstituted by halogen, e.g., C₁₋₆haloalkyl or C₁₋₄haloalkyl such as fluoromethyl and trifluoromethyl.

The term “heteroatom” as used herein means an atom of any element other than carbon or hydrogen. Preferred heteroatoms are nitrogen, oxygen, sulfur and phosphorus.

The terms “polycyclyl” or “polycyclic radical” refer to the radical of two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The term “isomers” or “stereoisomers” refers to compounds which have identical chemical constitution, but differ with regard to the arrangement of the atoms or groups in space.

The terms “isolated” or “substantially purified” are used interchangeably herein and refer to vitamin D₃ compounds in a non-naturally occurring state. The compounds can be substantially free of cellular material or culture medium when naturally produced, or chemical precursors or other chemicals when chemically synthesized. In one embodiment of the invention an isolated vitamin D compound is at least 75% pure, especially at least 85% pure, in particular at least 95% pure and preferably at least 99% pure on a w/w basis, said purity being by reference to compounds with which the vitamin D compound is naturally associated or else chemically associated in the course of chemical synthesis.

In certain preferred embodiments, the terms “isolated” or “substantially purified” also refer to preparations of a chiral compound which substantially lack one of the enantiomers; i.e., enantiomerically enriched or non-racemic preparations of a molecule.

Similarly, the terms “isolated epimers” or “isolated diastereomers” refer to preparations of chiral compounds which are substantially free of other stereochemical forms. For instance, isolated or substantially purified vitamin D₃ compounds include synthetic or natural preparations of a vitamin D₃ enriched for the stereoisomers having a substituent attached to the chiral carbon at position 3 of the A-ring in an alpha-configuration, and thus substantially lacking other isomers having a beta-configuration. Unless otherwise specified, such terms refer to vitamin D₃ compositions in which the ratio of alpha to beta forms is greater than 1:1 by weight. For instance, an isolated preparation of an epimer means a preparation having greater than 50% by weight of the alpha-epimer relative to the beta stereoisomer, more preferably at least 75% by weight, and even more preferably at least 85% by weight. Of course the enrichment can be much greater than 85%, providing “substantially epimer-enriched” preparations, i.e., preparations of a compound which have greater than 90% of the alpha-epimer relative to the beta stereoisomer, and even more preferably greater than 95%. The term “substantially free of the beta stereoisomer” will be understood to have similar purity ranges.

As used herein, the term “vitamin D compound” includes any compound being an analogue of vitamin D that is capable of treating or preventing prostate cancer. Generally, compounds which are ligands for the Vitamin D receptor (VDR ligands) and which are capable of treating or preventing prostate cancer are considered to be within the scope of the invention. Vitamin D compounds are preferably agonists of the vitamin D receptor. Thus, vitamin D compounds are intended to include secosteroids. Examples of specific vitamin D compounds suitable for use in the methods of the present invention are further described herein. A vitamin D compound includes vitamin D₂ compounds, vitamin D₃ compounds, isomers thereof, or derivatives/analogues thereof. Preferred vitamin D compounds are vitamin D₃ compounds which are ligands of (more preferably are agonists of) the vitamin D receptor. Preferably the vitamin D compound (e.g., the vitamin D₃ compound) is a more potent agonist of the vitamin D receptor than the native ligand (i.e., the vitamin D, e.g., vitamin D₃). Vitamin D, compounds, vitamin D₂ compounds and vitamin D₃ compounds include, respectively, vitamin D₁, D₂, D₃ and analogues thereof. In certain embodiments, the vitamin D compound may be a steroid, such as a secosteroid, e.g., calciol, calcidiol or calcitriol. Non-limiting examples of certain preferred vitamin D compounds in accordance with the invention include those described in U.S. Pat. No. 5,939,408 and U.S. Pat. No. 6,255,501.

As used herein, the term “obtaining” includes purchasing, synthesizing, isolating or otherwise acquiring one or more of the vitamin D compounds used in practicing the invention.

The term “secosteroid” is art-recognized and includes compounds in which one of the cyclopentanoperhydro-phenanthrene rings of the steroid ring structure is broken. For example, 1-alpha,25(OH)₂D₃ and analogues thereof are hormonally active secosteroids. In the case of vitamin D₃, the 9-10 carbon-carbon bond of the B-ring is broken, generating a seco-B-steroid. The official IUPAC name for vitamin D₃ is 9,10-secocholesta-5,7,10(19)-trien-3B-ol. For convenience, a 6-s-trans conformer of 1alpha,25(OH)₂D₃ is illustrated herein having all carbon atoms numbered using standard steroid notation.

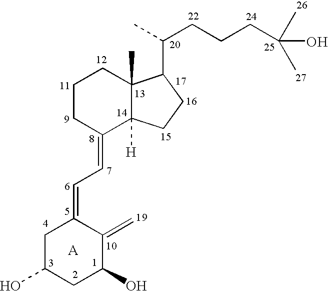

In the formulas presented herein, the various substituents on ring A are illustrated as joined to the steroid nucleus by one of these notations: a dotted line (----) indicating a substituent which is in the beta-orientation (i.e., above the plane of the ring), a wedged solid line [symbol] indicating a substituent which is in the alpha-orientation (i.e., below the plane of the molecule), or a wavy line [symbol] indicating that a substituent may be either above or below the plane of the ring. In regard to ring A, it should be understood that the stereochemical convention in the vitamin D field is opposite from the general chemical field, wherein a dotted line indicates a substituent on Ring A which is in an alpha-orientation (i.e., below the plane of the molecule), and a wedged solid line indicates a substituent on ring A which is in the beta-orientation (i.e., above the plane of the ring).

Furthermore the indication of stereochemistry across a carbon-carbon double bond is also opposite from the general chemical field in that “Z” refers to what is often referred to as a “cis” (same side) conformation whereas “E” refers to what is often referred to as a “trans” (opposite side) conformation. Regardless, both configurations, cis/trans and/or Z/E are contemplated for the compounds for use in the present invention.

As shown, the A ring of the hormone 1-alpha,25(OH)₂D₃ contains two asymmetric centers at carbons 1 and 3, each one containing a hydroxyl group in well-characterized configurations, namely the 1-alpha- and 3-beta-hydroxyl groups. In other words, carbons 1 and 3 of the A ring are said to be “chiral carbons” or “carbon centers.”

With respect to the nomenclature of a chiral center, terms “d” and “I” configuration are as defined by the IUPAC Recommendations. As to the use of the terms, diastereomer, racemate, epimer and enantiomer will be used in their normal context to describe the stereochemistry of preparations.

Also, throughout the patent literature, the A ring of a vitamin D compound is often depicted in generic formulae as any one of the following structures:

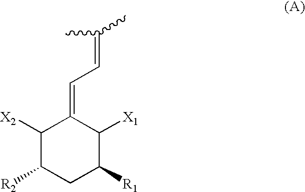

wherein X₁ and X₂ are defined as H or ═CH₂; or

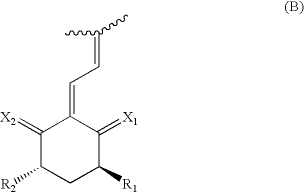

wherein X₁ and X₂ are defined as H₂ or CH₂.

Although there does not appear to be any set convention, it is clear that one of ordinary skill in the art understands either formula (A) or (B) to represent an A ring in which, for example, X₁ is ═CH₂ and X₂ is defined as H₂, as follows:

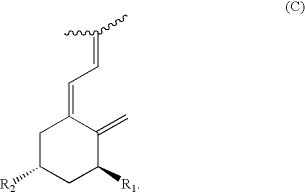

Those skilled in the art will recognise that the vitamin D compounds may be used in human or veterinary medicine. Thus, in accordance with the invention, the terms “subject” and “patient” are used interchangeably, and are intended to include mammals, for example, humans. It is preferred that the vitamin D compound be used in the treatment of human patients.

### Compounds, Pharmaceutical Compositions and Methods of Use

In one aspect, the invention provides a method for preventing or treating prostate cancer in a subject, comprising administering to a subject in need thereof a therapeutically effective amount of a vitamin D compound of formula (I):

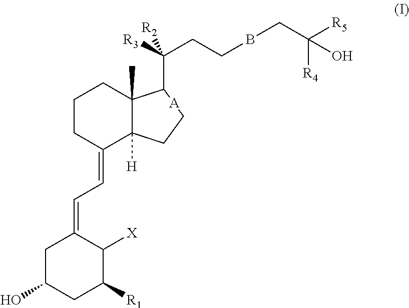

wherein:

### X is H₂ or CH₂;

R₁ is hydrogen, hydroxy or fluoro;
R₂ is hydrogen or methyl;
R₃ is hydrogen or methyl, wherein both R₂ and R₃ cannot both be hydrogen;
R₄ is methyl, ethyl or trifluoromethyl;
R₅ is methyl, ethyl or trifluoromethyl;
A is a single or double bond; and
B is a single, E-double, Z-double or triple bond; and pharmaceutically acceptable esters, salts, and prodrugs thereof.

In one embodiment the invention provides the use of compounds of formula (I) wherein A is a double bond. In another embodiment, B is an E-double bond. In yet another embodiment, X is CH₂.

In other embodiments, the invention provides the use of compounds of formula (I) wherein R₁ is fluoro. In another embodiment, R₂ is hydrogen. In yet another embodiment, R₃ is methyl. In still another embodiment, R₄ and R₅ are each ethyl.

In certain embodiments the invention provides the use of compounds of formula (I), wherein A is a double bond, B is a E-double bond, and X is CH₂. In a preferred embodiment R₁ is fluoro.

Other embodiments of the invention include the use of compounds of formula (I) wherein R₂ is hydrogen. In another embodiment, R₃ is methyl. In yet another embodiment, R₄ and R₅ are each ethyl.

In preferred compounds, each of R₄ and R₅ is methyl or ethyl, for example 1-alpha-fluoro-25-hydroxy-16,23E-diene-26,27-bishomo-20-epi-cholecalciferol (Compound 1) having the formula:

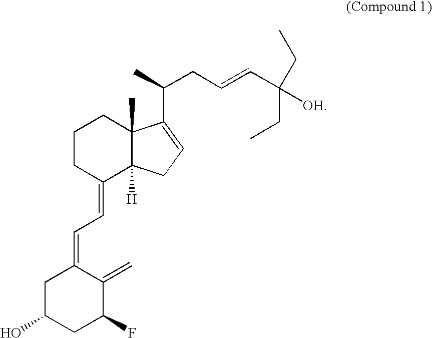

Such compounds are described in U.S. Pat. No. 5,939,408 and EP808833, the contents of which are herein incorporated by reference in their entirety.

It will be noted that the structures of some of the compounds of the invention include asymmetric carbon atoms. Accordingly, it is to be understood that the isomers arising from such asymmetry (e.g., all enantiomers and diastereomers) are included within the scope of this invention, unless indicated otherwise. Such isomers can be obtained in substantially pure form by classical separation techniques and/or by stereochemically controlled synthesis.

Naturally occurring or synthetic isomers can be separated in several ways known in the art. Methods for separating a racemic mixture of two enantiomers include chromatography using a chiral stationary phase (see, e.g., “Chiral Liquid Chromatography,” W. J. Lough, Ed. Chapman and Hall, New York (1989)). Enantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases such as brucine, quinine, ephedrine, strychnine, and the like. Alternatively, diastereomeric esters can be formed with enantiomerically pure chiral alcohols such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts.

The methods of the invention provide for the administration to subjects in need of prevention or treatment of prostate cancer of vitamin D compounds of formula (I) for the prevention or treatment of prostate cancer. In one embodiment the method further comprises identifying a subject in need of prevention or treatment for prostate cancer. In another embodiment, the method further comprises the step of obtaining the vitamin D compound of formula (I). In one embodiment, the subject is a mammal. In a preferred embodiment, the subject is a human. In other embodiments of the method, the vitamin D compound of formula (I) is formulated in a pharmaceutical composition together with a pharmaceutically acceptable diluent or carrier.

Suitably, the various aspects of the present invention are directed towards the treatment of prostate cancer. Alternatively, the various aspects of the present invention are directed towards the prevention of prostate cancer.

In another embodiment, the invention also provides a pharmaceutical composition, comprising an effective amount of a vitamin D compound as described herein and a pharmaceutically acceptable carrier. In a further embodiment, the effective amount is effective to treat prostate cancer, as described previously.

In an embodiment, the vitamin D compound is administered to the subject using a pharmaceutically-acceptable formulation, e.g., a pharmaceutically-acceptable formulation that provides sustained delivery of the vitamin D compound to a subject for at least 12 hours, 24 hours, 36 hours, 48 hours, one week, two weeks, three weeks, or four weeks after the pharmaceutically-acceptable formulation is administered to the subject.

In certain embodiments, these pharmaceutical compositions are suitable for topical or oral administration to a subject. In other embodiments, as described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension, (3) topical application, for example, as a cream, ointment or spray applied to the skin; (4) intrarectally, for example, as a pessary, cream or foam; or (5) aerosol, for example, as an aqueous aerosol, liposomal preparation or solid particles containing the compound.

The phrase “pharmaceutically acceptable” refers to those vitamin D compounds of the present invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminium hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing a vitamin D compound(s) include those suitable for oral, nasal, topical (including buccal and sublingual), rectal, aerosol and/or parenteral administration. The compositions may conveniently be presented in unit dosage form and may be prepared by any methods well known in the art of pharmacy.

The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these compositions include the step of bringing into association a vitamin D compound(s) with the carrier and, optionally, one or more accessory ingredients. In general, the formulations are prepared by uniformly and intimately bringing into association a vitamin D compound with liquid carriers, or finely divided solid carriers, or both, and then, if necessary, shaping the product.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a vitamin D compound(s) as an active ingredient. A compound may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes.

The active ingredient can also be in micro-encapsulated form, if appropriate, with one or more of the above-described excipients.

Liquid dosage forms for oral administration of the vitamin D compound(s) include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions, in addition to the active vitamin D compound(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal or administration may be presented as a suppository, which may be prepared by mixing one or more vitamin D compound(s) with one or more suitable nonirritating excipients or carriers comprising, for example, cocoa butter, polyethylene glycol, a suppository wax or a salicylate, and which is solid at room temperature, but liquid at body temperature and, therefore, will melt in the rectum and release the active agent.

Dosage forms for the topical or transdermal administration of a vitamin D compound(s) include powders, sprays, ointments, pastes, creams, lotions, gels, solutions, patches and inhalants. The active vitamin D compound(s) may be mixed under sterile conditions with a pharmaceutically-acceptable carrier, and with any preservatives, buffers, or propellants which may be required.

The ointments, pastes, creams and gels may contain, in addition to vitamin D compound(s) of the present invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a vitamin D compound(s), excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The vitamin D compound(s) can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a vitamin D compound(s) to the body. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel.

Pharmaceutical compositions of the invention suitable for parenteral administration comprise one or more vitamin D compound(s) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminium monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form.

Alternatively, delayed absorption of a parenterally-administered drug form is accomplished by dissolving or suspending the drug in an oil vehicle.

Injectable depot forms are made by forming microencapsule matrices of vitamin D compound(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The invention also provides kits for treatment or prevention of prostate cancer or a disease or disorder (or symptoms) thereof associated with prostate cancer. In one embodiment, the kit includes an effective amount of a compound in unit dosage form, together with instructions for administering the compound to a subject suffering from or susceptible to prostate cancer or a disease or disorder or symptoms thereof associated with prostate cancer, wherein the effective amount of compound is less than 500 mg of the compound.

In preferred embodiments, the kit comprises a sterile container which contains the compound; such containers can be boxes, ampules, bottles, vials, tubes, bags, pouches, blister-packs, or other suitable container form known in the art. Such containers can be made of plastic, glass, laminated paper, metal foil, or other materials suitable for holding medicaments.

The instructions will generally include information about the use of the compound for prevention or treatment of prostate cancer or a disease or disorder or symptoms thereof associated with prostate cancer; in preferred embodiments, the instructions include at least one of the following: description of the compound; dosage schedule and administration for treatment of a disease or disorder or symptoms thereof associated with prostate cancer; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

When the vitamin D compound(s) are administered as pharmaceuticals, to humans and animals, they can be given per se or as a pharmaceutical composition containing, for example, 0.1 to 99.5% (more preferably, 0.5 to 90%) of active ingredient in combination with a pharmaceutically-acceptable carrier.

Regardless of the route of administration selected, the vitamin D compound(s), which may be used in a suitable hydrated form, and/or the pharmaceutical compositions of the present invention, are formulated into pharmaceutically-acceptable dosage forms by conventional methods known to those of skill in the art.

Actual dosage levels and time course of administration of the active ingredients in the pharmaceutical compositions of the invention may be varied so as to obtain an amount of the active ingredient which is effective to achieve the desired therapeutic response for a particular patient, composition, and mode of administration, without being toxic to the patient. An exemplary dose range is from 0.1 to 300 μg per day

A preferred dose of the vitamin D compound for the present invention is the maximum that a patient can tolerate and not develop hypercalcemia. Preferably, the vitamin D compound of the present invention is administered at a concentration of about 0.001 ug to about 100 ug per kilogram of body weight, about 0.001-about 10 ug/kg or about 0.001 ug-about 100 ug/kg of body weight. Ranges intermediate to the above-recited values are also intended to be part of the invention.

The vitamin D compound may be administered separately, sequentially or simultaneously in separate or combined pharmaceutical formulations with a second medicament for the treatment of prostate cancer (for example a second vitamin D compound of the present invention).

In certain embodiments of the methods, pharmaceutical compositions, or kits of the invention, the vitamin D compound is administered separately, sequentially or simultaneously in separate or combined pharmaceutical formulations with a further medicament for the treatment or prevention of prostate cancer. In one embodiment, the further medicament is an alpha-adrenergic receptor blocking agent. The alpha-adrenergic receptor blocking agent includes but is not limited to terazosin, doxazosin, tamsulosin, silodosin, AIO-8507L and RBx-2258.

In another embodiment, the further medicament is a 5 alpha-reductase inhibitor. In specific embodiments, the 5 alpha-reductase inhibitors include but are not limited to finasteride and dutasteride.

Suitably the further medicament is indicated for the treatment of prostate cancer.

In preferred embodiments of the methods, pharmaceutical compositions, or kits according to any preceding claim, the vitamin D compound, or pharmaceutically acceptable composition or formulation thereof is provided in unit dose form. Preferably, the unit dose of the vitamin D compound is 50 to 150 ug.

The methods, pharmaceutical compositions, or kits of the invention are particularly advantageous in that the vitamin D compounds of the invention provide for the prevention or treatment of prostate cancer without anti-androgenic prostatic and extra-prostatic adverse effects.

### Synthesis of Compounds of the Invention

The syntheses of compounds of the invention have been described in the art, for example in U.S. Pat. No. 5,939,408 and U.S. Pat. No. 6,255,501, the contents of which are incorporated herein by reference in their entirety.

The synthesis of the vitamin D₃ analogue Compound 1, shown below in Scheme 1, has been previously reported in the literature (Radinov et al. J. Org. Chem. (2001), 66, 6141; Daniewski et al. U.S. Pat. No. 6,255,501; Batcho et al. U.S. Pat. No. 5,939,408). In general the prior art synthesis of vitamin D₃ analogue 1 requires 28 process steps. However, Schemes 2-4 below provide a simplified synthesis of vitamin D₃ analogue 1, in 19-21 steps.

As shown in Schemes 1-4, the synthesis of vitamin D₃ analogue 1 includes starting material cleavage, allylic oxidation, rearrangements, chain length extension, selective 1,2-addition, and Horner-Wittig coupling. Although the synthesis of compounds of use in the present invention is described by reference to Schemes 1-4, which exemplify the synthesis of vitamin D₃ analogue 1, a number of other vitamin D₃ can be synthesized using the methods described in this section and the following working examples without undue experimentation.

Scheme 1 provides a summary of the conversion of vitamin D₂ (2) to compound 1. Compound 2 was initially hydroxyl protected. Oxidation with ozone, followed by a reductive workup provided intermediates 3 and 4. The conversion of 4 to 6 took place over eight steps, and included olefin epoxidation, allylic oxidation, and deoxygenation. The conversion of 3 to 5 was accomplished over eight steps and included allylic oxidation and rearrangement, and chain elongation. The final coupling of 5 and 6 took place under standard Horner-Wittig conditions to complete the novel synthesis of 1.

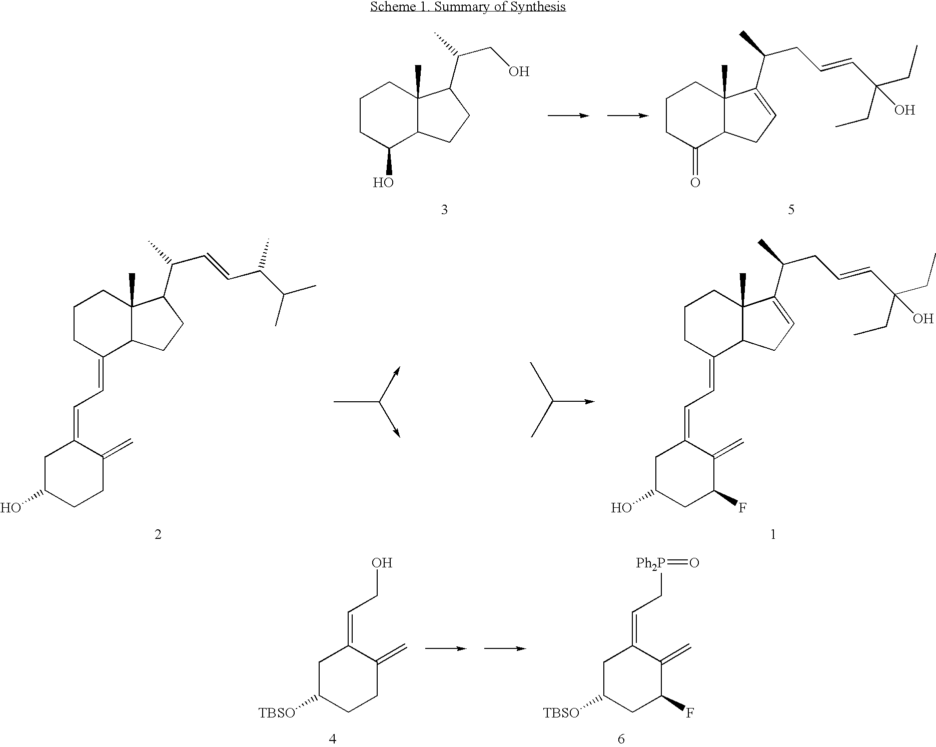

Scheme 2 outlines the cleavage of compound 2 to synthetic precursors 3 and 4. The hydroxyl group of 2 was initially protected with a t-butyl dimethyl silyl group, and ozonolysis was followed by a reductive workup with sodium borohydride to provide diol 3 in 60% yield, and alcohol 4 in 40% yield.

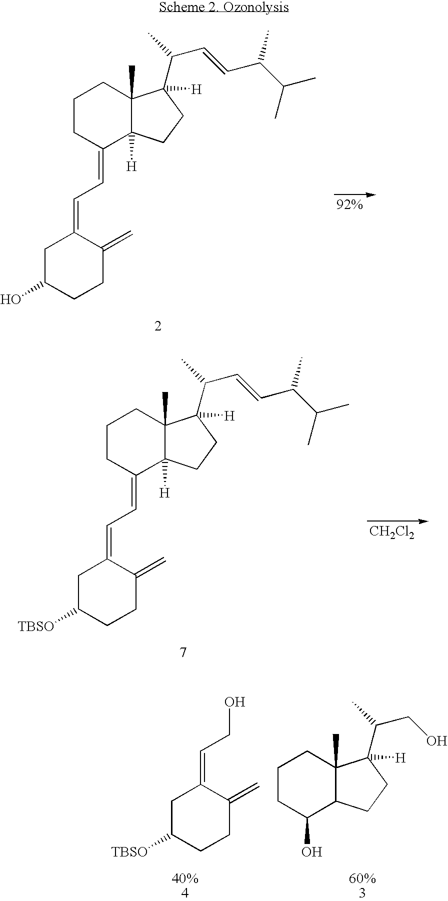

Scheme 3 details the conversion of 4 to the A-ring phosphine oxide 6. Compound 4 was epoxidized at the trisubstituted olefin in the presence of mCPBA in methylene chloride to provide 8 in 84% yield. Benzoyl protection of the primary hydroxyl group provided compound 9 in 91% yield, and was followed by allylic oxidation in the presence of selenium dioxide and t-butyl hydrogen peroxide in dioxane to give 10 as a mixture of epimeric compounds. The preferred isomer was reacted with diethylaminosulfur trifluoride (DAST) to provide fluorinated 11 in 75% yield. The conversion of 11 to 12 was accomplished in 61% yield in the presence of tris(3,5-dimethylpyrazoyl)hydridoborate rhenium trioxide and triphenyl phosphine in a sealed tube at 100° C. over 14 h. Benzyl hydrolysis in sodium methoxide solution provided hydroxyl compound 13 in 73% yield. The hydroxyl group of 13 was converted to the chloride compound 21 in the presence of triphosgene and pyridine, and subsequently converted to the Horner-Wittig reagent 6 by substitution of the chloride with diphenyl phosphine oxide. The conversion of 13 to 6 was accomplished in 76% yield.

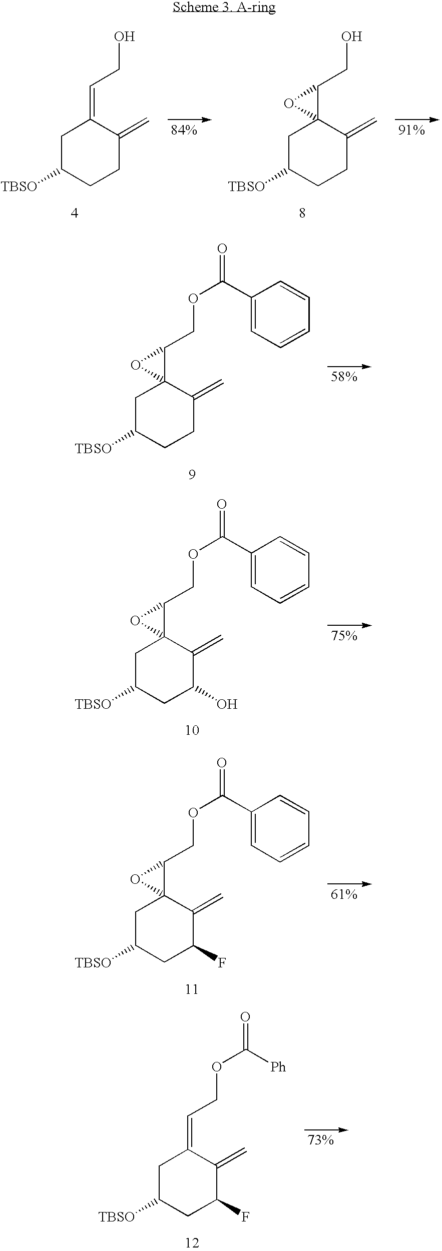

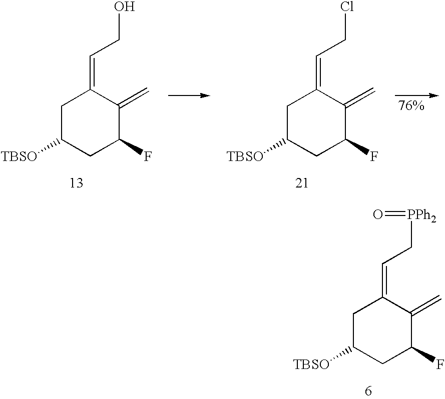

Scheme 4 describes the conversion of diol 3 to precursor 5. Compound 3 was oxidized to aldehyde 14 in 89% yield in the presence of TEMPO and NCS. The hydroxyl group was acetate protected to provide 15, and converted to the alkene mixture 16 in the presence of palladium and benzalacetone. Allylic oxidation provided an isomeric mixture of alcohols 17, which was subsequently subjected to Claisen rearrangement conditions to produce aldehyde 18 in 60% yield. Surprisingly, both isomers of 17 provided one isomer of 18. Chain elongation via a Wittig-Horner coupling provided ester 19 in high yield. Reduction of the ester with ethyl grignard in the presence of cerium trichloride provided diol 20 in 99% yield. The oxidation of 20 in the presence of PDC provided intermediate 5.

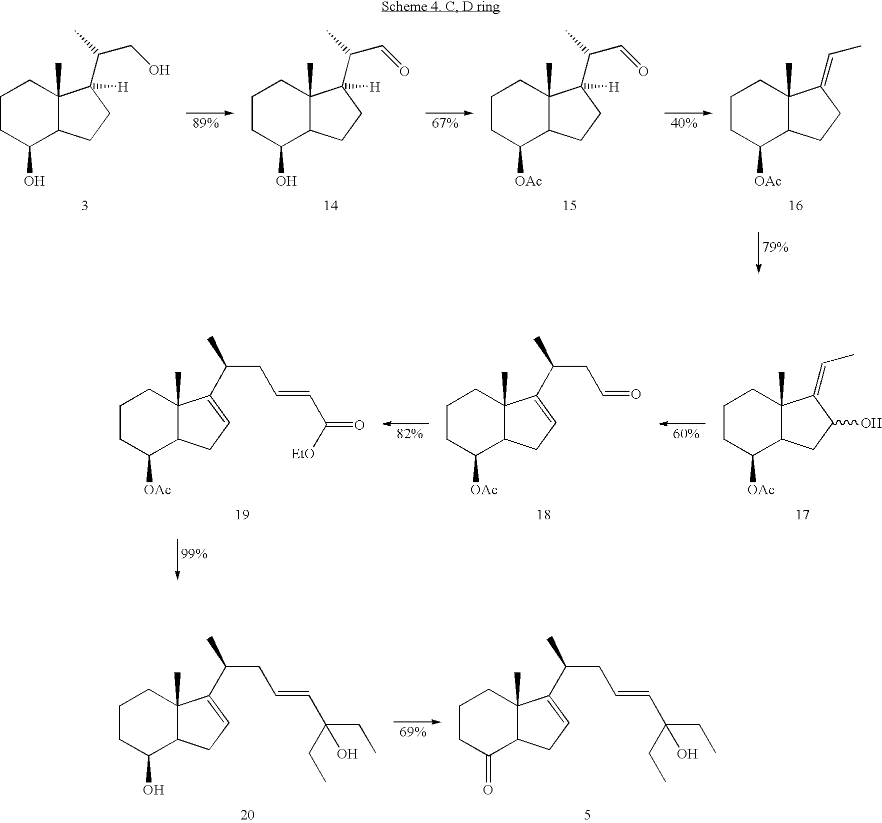

The conversion of 15 to 16 (scheme 4) was accomplished, although a number of olefin side products were observed. Since purification of 16 is tedious and requires the use of medium pressure silver nitrate impregnated silica gel column chromatography, the product mixture was utilized in the next step. The reaction mixture was subsequently subjected to oxidation conditions, wherein compound 17 and other oxidation products could be separated by column chromatography. Interestingly, the over-oxidized side product (ketone) could be converted to 17 by reaction with a reducing agent, notably NaBH₄.

In one embodiment, compound 5 was further protected with a trimethyl silyl group, and then coupled with 6 in the presence of base (Scheme 5). The silyl protecting groups were removed in the presence of tetrabutyl ammonium fluoride (TBAF) to afford 1. The yield of 1 was 74% starting from the silyl protected 5. In another embodiment, compound 5 was coupled with 6 in the presence of base, followed by in situ deprotection of the silyl group with tetrabutyl ammonium fluoride (TBAF) to afford 1 (Scheme 5). The second embodiment therefore provides a one-step, one-pot synthesis of 1 starting from 5 and 6.

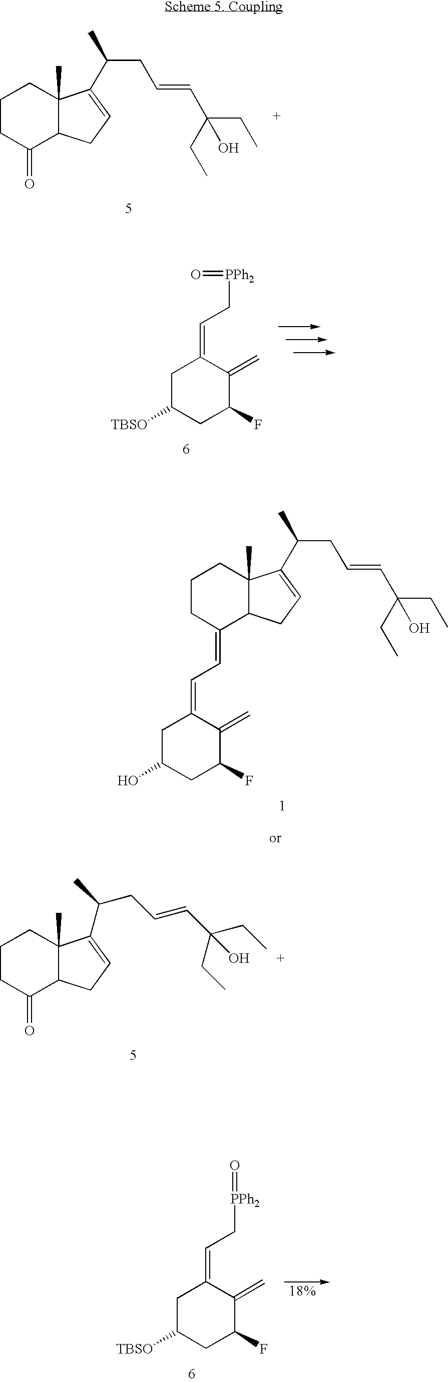

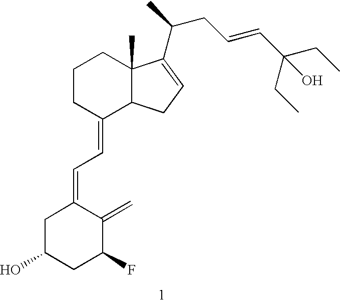

The invention therefore provides for the conversion of a compound 3 to a compound 5 (CD-ring portion) in eight steps. Additionally, seven of the eight steps provide reaction products in yields of 60-99%, demonstrating the efficacy of the synthetic route. The invention also provides the A-ring portion in eight steps starting from the vitamin D₂ cleavage product 4. Including the coupling steps of 5 and 6, the invention provides for a novel 19-step synthesis of 1. Alternatively, the invention also provides for a 21-step synthesis of 1. The current methodology represents a significant simplification of the protocol described and practiced previously which required 28 steps.

Chiral syntheses can result in products of high stereoisomer purity. However, in some cases, the stereoisomer purity of the product is not sufficiently high. The skilled artisan will appreciate that the separation methods described herein can be used to further enhance the stereoisomer purity of the vitamin D₃-epimer obtained by chiral synthesis.

Naturally occurring or synthetic isomers can be separated in several ways known in the art. Methods for separating a racemic mixture of two enantiomers include chromatography using a chiral stationary phase (see, e.g., “Chiral Liquid Chromatography,” W. J. Lough, Ed. Chapman and Hall, New York (1989)). Enantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases such as brucine, quinine, ephedrine, strychnine, and the like. Alternatively, diastereomeric esters can be formed with enantiomerically pure chiral alcohols such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts.

### EXEMPLIFICATION OF THE INVENTION

The present invention will now be described with reference to the following non-limiting examples.

### Synthesis of Compounds of the Invention

### Experimental

All operations involving vitamin D₃ analogs were conducted in amber-colored glassware in a nitrogen atmosphere. Tetrahydrofuran was distilled from sodium-benzophenone ketyl just prior to its use and solutions of solutes were dried with sodium sulfate. Melting points were determined on a Thomas-Hoover capillary apparatus and are uncorrected. Optical rotations were measured at 25° C. ¹H NMR spectra were recorded at 400 MHz in CDCl₃ unless indicated otherwise. TLC was carried out on silica gel plates (Merck PF-254) with visualization under short-wavelength UV light or by spraying the plates with 10% phosphomolybdic acid in methanol followed by heating. Flash chromatography was carried out on 40-65 um mesh silica gel. Preparative HPLC was performed on a 5×50 cm column and 15-30 um mesh silica gel at a flow rate of 100 mL/min.

### Chemical Example

### Synthesis of 1-alpha-Fluoro-25-hydroxy-16-23E-diene-26,27-bishomo-20-epi-cholecalciferol (1)

### Cleavage of the Vitamin D2 Starting Material

### t-Butyl-dimethyl-(4-methylene-3-{2-[7a-methyl-1-(1,4,5-trimethyl-hex-2-enyl)-octahydro-inden-4-ylidene]-ethylidene}-cyclohexyloxy)-silane (7)

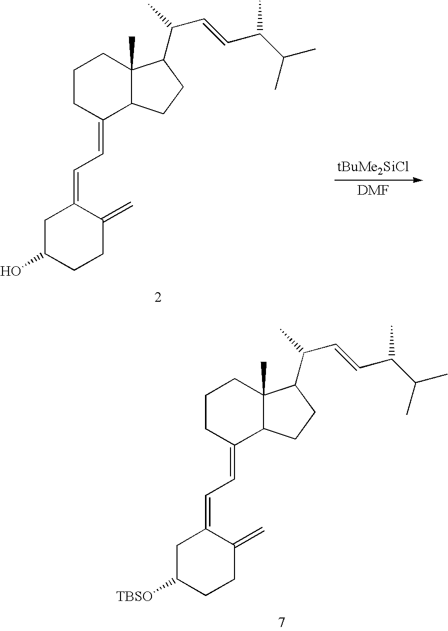

To a stirred solution of 2 (100.00 g, 0.25 mol) in DMF (250 mL), imidazole (40.80 g, 0.6 mol) and (t-butyldimethyl)silyl chloride (45.40 g, 0.3 mol) were added successively. The reaction mixture was stirred at room temperature for 1 h, diluted with hexane (750 mL), washed with water (500 mL),1 N HCl (500 mL), brine (500 mL) and dried over Na₂SO₄. The residue (155 g) after evaporation of the solvent was filtered through a plug of silica gel (500 g, 5% AcOEt in hexane) to give the title compound (115.98 g, 0.23 mol. 92%).

¹H-NMR: delta 0.04 and 0.08 (2s, 6H), 0.59 (s, 3H), 0.90 (d, 3H, J=6.6 Hz), 0.92 (d, 3H, J=6.6 Hz), 0.98 (s, 9H), 0.99 (d, 3H, J=7.0 Hz), 1.06 (d, 3H, J=6.8 Hz), 1.10-2.95 (m, 21H), 5.11 (br s, 2H), 5.22 (m, 2H), 6.49 (br s, 2H).

### 2-[(5-(tert-Butyl-dimethyl-silanyloxy)-2-methylene-cyclohexylidene]-ethanol (4) and 1-(2-Hydroxy-1-methyl-ethyl)-7a-methyl-octahydro-inden-4-ol (3)

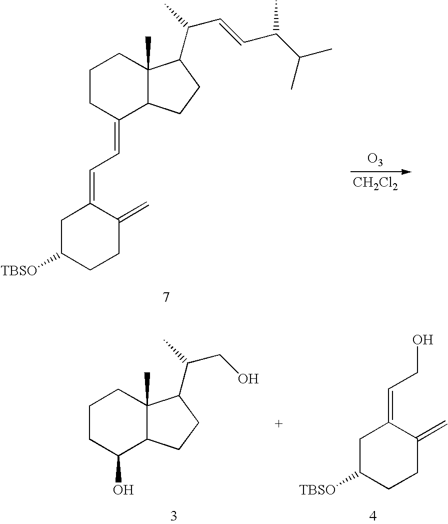

A stream of ozone was passed through a stirred solution of 7 (23.4 g, 45.8 mmol), pyridine (5.0 mL) and Sudane Red 7B (15.0 mg) in dichloromethane (550 mL), at −55 to −60° C. until Sudane Red decolorized (55 min). Sodium borohydride (6.75 g, 180 mmol) was then added followed by ethanol (250 mL). The reaction was allowed to warm to room temperature and stirred at room temperature for 1 h. Acetone (15 mL) was added and, after 30 min brine (300 mL) was added. The mixture was diluted with ethyl acetate (500 mL) and washed with water (600 mL). The aqueous phase was extracted with AcOEt (300 mL). The combined organic phases were dried over Na₂SO₄. The residue (26.5 g), after evaporation of the solvent, was filtered through a plug of silica gel (500 g, 15%, 30% and 50% AcOEt in hexane) to give:

Fraction A (5.9 g, mixture containing the desired A-ring (ca 83% pure by NMR) ¹H NMR: delta 5.38 (1H, t, J=6.4 Hz), 4.90 (1H, brs), 4.57 (1H, brs), 4.22 (1H, dd, J=7.3, 12.5 Hz), 4.13 (1H, dd, J=6.3, 12.5 Hz), 3.78 (1H, m), 2.40-1.30 (6H, m), 0.83 (9H, s), 0.01 (3H, s), 0.00 (3H, s); Fraction A was used for the synthesis of the A-ring precursor.
Fraction B (14.6 g, mixture containing a CD-rings fragments on a different stage of oxidation).
Fraction B was further ozonolyzed in order to obtain the Lythgoe diol (3). A stream of ozone was passed through a stirred solution of Fraction B (14.6 g) and Sudane Red 7B (3.0 mg) in ethanol (225 mL) at −55 to −60° C. for 30 min (Sudane Red decolorized). Sodium borohydride (3.75 g, 100 mmol) was added and the reaction was allowed to warm to room temperature and stirred at room temperature for 1 h. Acetone (5 mL) was added and, after 30 min brine (200 mL) was added. The mixture was diluted with dichloromethane (300 mL) and washed with water (250 mL). The aqueous phase was extracted with dichloromethane (200 mL). The combined organic phases were, evaporated to dryness (the last portion was evaporated with addition of toluene 100 mL). The residue (16.2 g) was dissolved in dichloromethane (100 mL), concentrated to a volume of ca 20 mL diluted with petroleum ether (30 mL) and set aside in the fridge for crystallization. The white powder was filtered of (4.05 g), the mother liquor was concentrated and filtered through silica gel (100 g, 5% MeOH in CH₂Cl₂) to give yellow oil (9.4 g), which was recrystallized (20 mL, dichloromethane; petroleum ether 1:2) to give white powder (1.79 g). Thus the total yield of the Lythgoe diol 3 was (5.84 g, 27.5 mmol, 60% from D₂) ¹H NMR: delta 4.08 (1H, m), 3.64 (1H, dd, J=3.3, 10.6 Hz), 3.39 (1H, dd, J=6.6, 10.6 Hz), 2.04-1.14 (15H, m), 1.03 (3H, d, J=6.6 Hz), 0.96 (3H, s).

### Synthesis of the A-Ring Precursor

### (2R,3S,7S)-[7-(t-butyldimethyl)silanyloxy)-4-methylene-1-oxa-spiro[2.5]oct-2-yl]-methanol (8)

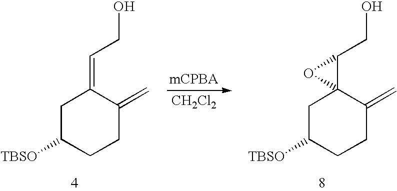

To a stirred solution of a crude 4 (5.9 g, ca 18.3 mmol, Fraction A from ozonolsysis) in dichloro-methane (120 mL) at room temperature, AcONa (2.14 g, 26.1 mmol) was added followed by 72% mCPBA (4.32 g, 18.0 mmol). The reaction mixture was then stirred at 10° C. for ½ h then diluted with hexane (200 mL) washed with 10% K₂CO₃ (3×150 mL), and dried over Na₂SO₄. The residue after evaporation of solvent (6.6 g) was filtered through a plug of silica gel (150 g, 10% AcOEt in hexane) to give the crude title compound (4.87 g, ca 15.4 mmol, 84%) ¹H-NMR: delta 0.063 and 0.068 (2s, 6H), 0.88 (s, 9H), 1.38-1.49 (m, 1H), 1.54 (m, 1H, OH), 1.62 (m, 1H), 1.96 (m, 3H), 2.43 (m, 1H), 3.095 (t, 1H, J=5.6 Hz), 3.60 (m, 2H), 3.86 (m, 1H), 4.91 (m, 1H).

### Benzoic acid (2R,3S,7S)-7-(t-butyldimethyl)silanyloxy)-4-methylene-1-oxa-spiro[2.5]oct-2-yl methyl ester (9)

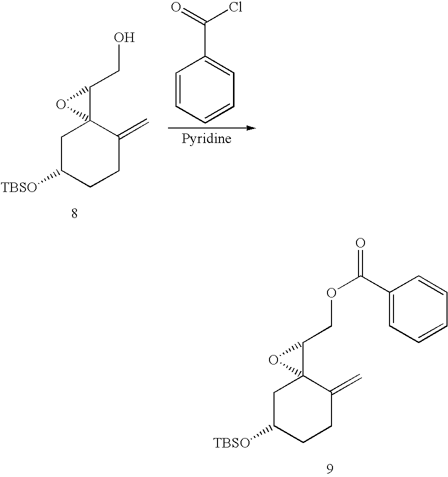

To a stirred solution of 8 (4.87 g, ca 15.4 mmol) in pyridine (25 mL) at room temperature, benzoyl chloride (2.14 mL, 18.4 mmol) was added and the reaction mixture was stirred for 1 h. Water (25 mL) was added and after stirring for 45 min at room temperature the mixture was diluted with hexane (80 mL), washed with saturated NaHCO₃ solution (50 mL), and dried over Na₂SO₄. The residue after evaporation of solvent (17.5 g) was purified by FC (150 g, 10% AcOEt in hexane) to give the title compound (5.44 g, 14.0 mmol,91%) ¹H NMR: delta 8.04-7.80 (2H, m), 7.56-7.50 (1H, m), 7.44-7.37 (2H, m), 4.94 (1H, brs), 4.92 (1H, brs), 4.32 (1H, dd, J=4.8, 11.9 Hz), 4.14 (1H, dd, J=6.2, 11.9 Hz), 3.83 (1H, m), 3.21 (1H, dd, J=4.8, 6.2 Hz), 2.42 (1H, m), 2.04-1.90 (3H, m), 1.64-1.34 (2H, m), 0.83 (9H, s), 0.02 (3H, s), 0.01 (3H, s).

### Benzoic acid (2R,3S,5R,7S)-7-(t-butyldimethyl)silanyloxy)-5-hydroxy-4-methylene-1-oxa-spiro[2.5]oct-2-yl methyl ester (10)

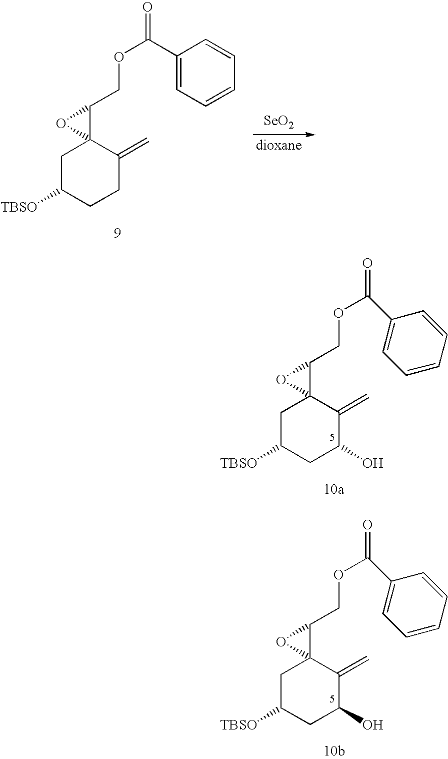

To a stirred solution of 9 (10.0 g, 25.7 mmol)) in dioxane (550 mL) at 85° C. was added selenium dioxide, (3.33 g, 30.0 mmol) followed by t-butyl hydrogen peroxide (9.0 mL, 45.0 mmol, 5-6 M in nonane) and the reaction mixture was stirred at 85° C. for 16 h, after which selenium dioxide (1.11 g, 10.0 mmol) was added followed by t-butyl hydrogen peroxide (3.0 mL, 15.0 mmol, 5-6 M in nonane) and the reaction mixture was stirred at 85° C. for additional 6 h. The solvent was removed under vacuum and the residue (15.3 g) was filtered through a plug of silica gel (300 g, 20% AcOEt in hexane) to give: starting material (970 mg, 10%) and a mixture of 10a and 10b (8.7 g). This mixture was divided into 3 portion (2.9 g each) and purified twice by FC (200 g, 5% isopropanol in hexane, same column was used for all six chromatographs) to give: 10b (1.83 g, as a 10:1 mixture of 10b:10a ca 16% of 5alpha-hydroxy compound); 10a (6.0 g, 14.8 mmol, 58%) as white crystals. The structure of 10a was confirmed by X-ray crystallography.

¹H NMR: delta 8.02-7.90 (2H, m), 7.58-7.50 (1H, m), 7.46-7.38 (2H, m), 5.25 (1H, br s), 5.11 (1H, brs), 4.26 (1H, dd, J=5.5, 12.1 Hz), 4.15 (1H, dd, J=5.9, 12.1 Hz), 4.07 (1H, m), 3.87 (1H, m), 3.19 (1H, dd, J=5.5, 5.9 Hz), 2.34-1.10 (5H, m), 0.81 (9H, s), 0.01 (3H, s), 0.00 (3H, s).

### Benzoic acid (2R,3S,5S,7R)-7-(t-butyldimethyl)silanyloxy)-5-fluoro-4-methylene-1-oxa-spiro[2.5]oct-2-ylmethyl ester (11)

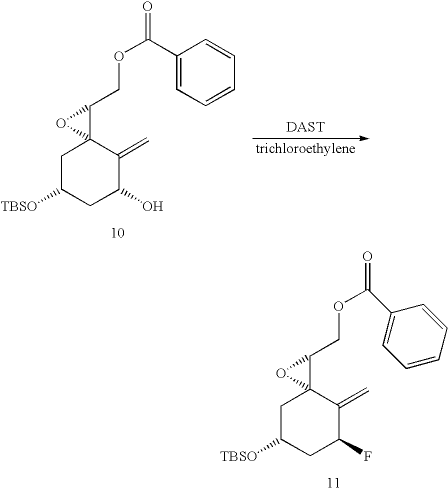

To a stirred solution of a diethylaminosulfur trifluoride (DAST) (2.0 mL, 16.0 mmol) in trichloroethylene (20 mL) a solution of 10 (2.78 g, 6.87 mmol) in trichloroethylene (126 mL was added at −75° C. After stirring for 20 min at −75° C. methanol (5.5 mL) was added followed by saturated NaHCO₃ solution (6 mL) and the resulting mixture was diluted with hexane (150 mL) and washed with saturated NaHCO₃ solution (100 mL), dried over Na₂SO₄ and concentrated. The residue (4.5 g) was purified by FC (150 g, DCM:hexane:AcOEt 10:20:0.2) to give the title compound (2.09 g, 5.14 mmol, 75%) ¹H NMR: delta 8.02-7.99 (2H, m), 7.53-7.45 (1H, m), 7.40-7.33 (2H, m), 5.26 (2H, m), 5.11 (1H, dt, J=3.0, 48.0 Hz), 4.46 (1H, dd, J=3.3, 12.5 Hz), 4.21 (1H, m), 3.94 (1H, dd, J=7.7, 12.5 Hz), 3.29 (1H, dd, J=3.3, 7.7 Hz), 2.44-1.44 (4H, m), 0.80 (9H, s), 0.01 (3H, s), 0.00 (3H, s).

### Benzoic acid 2-[5-(tert-butyl-dimethyl-silanyloxy)-3-fluoro-2-methylene-cyclohexylidene]-ethyl ester (12)

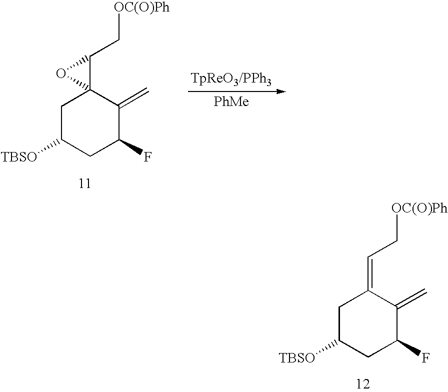

A mixture of tris(3,5-dimethylpyrazoyl)hydridoborate rhenium trioxide (265 mg, 0.50 mmol), triphenylphosphine (158 mg, 0.6 mmol), epoxide 11 (203 mg, 0.5 mmol) and toluene (8 mL) was sealed in an ampule under argon and heated at 100° C. for 14 h. (TLC, 10% AcOEt in hexane, mixture of substrate and product, ca 1:1). Rhenium oxide did not completely solubilized. A solution of triphenylphosphine (158 mg, 0.6 mmol) in toluene (4 mL) was added and the heating continued for 6 h. The reaction mixture was cooled to room temperature filtered through a plug of silica gel and then the residue after evaporation of the solvent was purified by FC (20 g, 5% AcOEt in hexane) to give: 12 (120 mg, 0.31 mmol, 61% of the desire product) and 70 mg of the starting material plus minor contaminations, ca 34%.

### (1Z,3S,5R)-2-[5-(t-butyldimethyl)silanyloxy)-3-fluoro-2-methylene-cyclohexylidene]-ethanol (13)

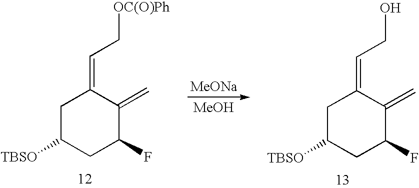

To a solution of a benzoate 12 (150 mg, 0.38 mmol) in methanol (3 mL) was added sodium methoxide (0.5 mL, 15% in methanol). After stirring for 1 h at room temperature water was added (6 mL) and the mixture was extracted with methylene chloride (3×10 mL). The combined organic layers was dried over Na₂SO₄ and evaporated to dryness. The residue (0.2 g) was purified by FC (20 g, 15% AcOEt in hexane) to give 13 (80 mg, 0.28 mmol, 73% of the product)

### (1R,3Z,5S)-t-butyl-[3-(2-chloro-ethylidene)-5-fluoro-4-methylene-cyclohexyloxy]-dimethylsilane (21)

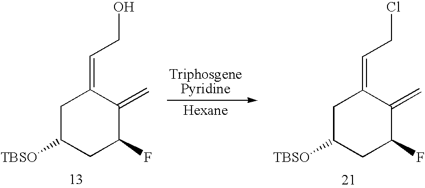

To a solution of 13 (8.07 g, 28.2 mmol) and triphosgene (4.18 g, 14.1 mmol) in hexane (150 mL) at 0° C. was added over 30 min a solution of pyridine (4.5 mL, 55.6 mmol) in hexane (20 mL) and the reaction mixture was stirred at this temperature for 30 min and at room temperature for another 30 min. The reaction mixture was washed with CuSO₄ aq (3×200 mL). The combined aqueous layers were back-extracted with hexane (2×100 mL). The organic layers were combined, dried (MgSO₄), and concentrated in vacuo to give the title compound (9.0 g, overweight). This material was used immediately in the next step without further purification. [alpha]²⁵D+73.0° (c 0.28, CHCl₃); IR (CHCl₃) 1643, 838 cm⁻¹; ¹H-NMR delta 0.08 (s, 6H), 0.88 (s, 9H), 1.84-2.03 (m, 1H), 2.12 (brs, 1H), 2.24 (m, 1H), 2.48 (brd, J=13 Hz, 1H), 4.06-4.26 (m, 3H), 5.10 (br d, J=48 Hz), 5.16 (s, 1H), 5.35 (s, 1H), 5.63 (br t, J=6 Hz, 1H).

### (1S,3Z,5R)-1-fluoro-5-(t-butyldimethyl)silanyloxy)-2-methenyl-3-(diphenylphosphinoyl)ethylidene cyclohexane (6)

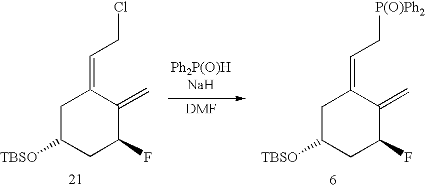

Diphenylphosphine oxide (6.70 g, 33.1 mmol) was added portionwise, over 15 min to a suspension of NaH (1.33 g, 33.1 mmol, 60% dispersion in mineral oil) in DMF (50 mL) at 10° C. The resulting solution was stirred at room temperature for 30 min and cooled to −60° C. The solution of crude 21 (9.0 g) in DMF (20 mL) was then added dropwise. The reaction mixture was stirred at −60° C. for 2 h and at room temperature for 1 h, diluted with diethyl ether (600 mL) and washed with water (3×200 mL). The aqueous layers were extracted with diethyl ether (200 mL). The combined organic layers were dried (MgSO₄) and concentrated under reduced pressure to give white solid. The crude product was recrystallized from diisopropyl ether (25 mL). The resulting solid was collected by filtration, washed with cold diisopropyl ether (5 mL) and dried under high vacuum to give the title compound (7.93 g). The mother liquor was concentrated and the residue was subjected to chromatography on silica gel (50 g, 30%-50% AcOEt in hexane) to give title compound (2.22 g). Thus the total yield of the of 6 was (10.1 g, 21.5 mmol, 76% overall from 13. [alpha]²⁵_{D}+50.2° (c 0.84, CHCl₃); IR (CHCl₃) 835, 692 cm⁻¹; UVλₘₐₓ (ethanol) 223 (ε 22770), 258 (1950), 265 (1750), 272 nm (1280); MS, m/e 470 (M⁺), 455 (4), 450 (8), 413 (98), 338 (9), 75 (100); ¹H-NMR: delta 0.02 (s, 6H), 0.84 (s, 9H), 1.76-1.93 (m, 1H), 2.16 (m, 2H), 2.42 (br d, 1H), 3.28 (m, 2H), 4.01 (m, 1H), 5.02 (dm, J=44 Hz, 1H), 5.14 (s, 1H), 5.30 (s, 1H), 5.5 (m, 1H), 7.5 (m, 6H), 7.73 (m, 4H). Analysis Calcd for C₂₇H₃₆O₂FPSi: C, 68.91; H, 7.71; F, 4.04. Found: C, 68.69; H, 7.80; F, 3.88.

### Synthesis of C,D-Ring/Side Chain Precursor

### (S)-2-((1R,3aR,4S,7aR)-4-hydroxy-7a-methyl-octahydro-inden-1-yl)-propionaldehyde (14)

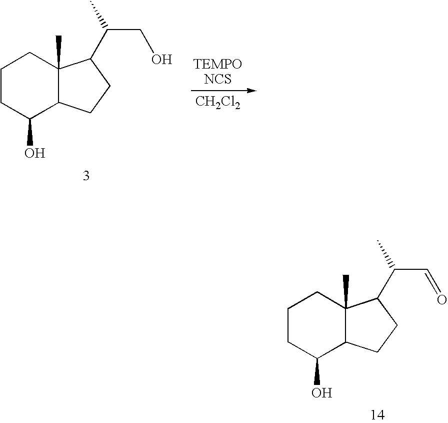

A 250-mL flask was charged with 0.99 g (4.67 mmol) of Lythgoe diol (3), 75 mg (0.48 mmol) of TEMPO, 146 mg (0.53 mmol) of tetrabutylammonium chloride hydrate, and dichloromethane (50 mL). To this vigorously stirred solution was added a buffer solution (50 mL) prepared by dissolving sodium hydrogen carbonate (4.2 g) and potassium carbonate (0.69 g) in a volume of 100 mL of water. The mixture was stirred vigorously and 839 mg (6.28 mmol) of N-chlorosuccinimide was added. TLC (1:2, ethyl acetate-heptane) showed the gradual conversion of educt (Rf 0.32) to the aldehyde 14 (Rf 0.61). After 18 h an additional quantity of 830 mg (6.28 mmol) of N-chlorosuccinimide was added and one hour later 20 mg of TEMPO was added and the mixture was stirred for 24 h. The organic layer was separated and the aqueous layer re-extracted with dichloromethane (3×50 mL). The combined organic extracts were washed with brine, dried and concentrated in vacuo. The residue was purified by column chromatography (SiO₂, ethyl acetate/heptane=1:3) to furnish 876 mg of crude aldehyde 14 (89%) ¹H NMR: delta 9.58 (1H, d, J=2.8 Hz), 4.12 (1H, m), 2.50-2.30 (1H, m), 2.10-1.10 (13H, m), 1.11 (3H, d, J=7.0 Hz), 0.99 (3H, s).

### (1R,3aR,4S,7aR)-7a-methyl-1-((S)-1-methyl-2-oxo-ethyl)-octahydroinden-4-yl ester (15)

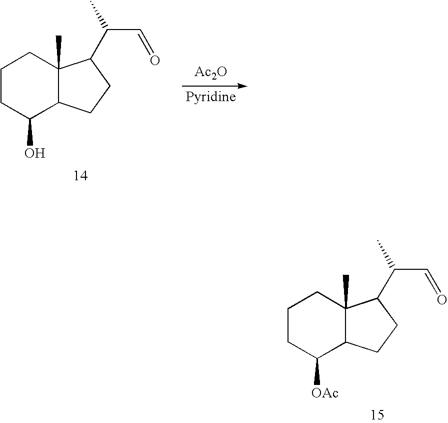

The crude 14 (255 mg, 1.21 mmol) was dissolved in pyridine (1 mL), the soln. cooled in an ice bath and DMAP (5 mg) and acetic anhydride (0.5 mL) were added. The mixture was stirred at room temperature for 24 h then diluted with water (10 mL), stirred for 10 min and equilibrated with ethyl acetate (30 mL). The organic layer was washed with a mixture of water (10 mL) and 1 N sulfuric acid (14 mL), then with water (10 mL) and saturated sodium hydrogen carbonate solution (10 mL), then dried and evaporated. The resulting residue (201 mg) was chromatographed on a silica gel column using 1:4 ethyl acetate-hexane as mobile phase. The fractions containing the product were pooled and evaporated to give the title compound as a colorless syrup (169 mg, 0.67 mmol, 67%). ¹H NMR (300 MHz, CDCl₃): delta 9.56 (1H, d, J=2.0 Hz), 5.20 (1H, brs), 2.44-2.16 (1H, m), 2.03 (3H, s), 2.00-1.15 (12H, m), 1.11 (3H, d, J=7.0 Hz), 0.92 (3H, s).

### Acetic acid (3aR,4S,7aR)-1-E-ethylidene-7a-methyl-octahydroinden-4-yl ester (16)

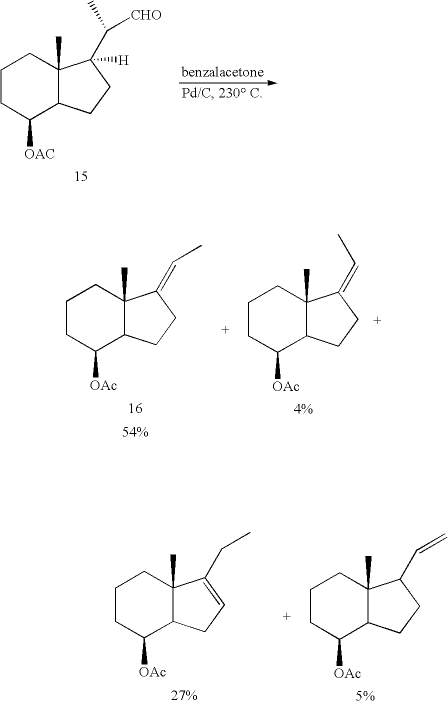

To a solution of aldehyde 15 (480 mg, 1.90 mmol) in diethylether (5 mL) was added 10% Pd on Carbon (25 mg). The suspension was stirred at room temperature for 20 min., filtered through a path of Celite and the filtrate was concentrated in vacuo. To the residue was added benzalacetone (350 mg, 2.40 mmol, distilled) and 10% Pd on Carbon (50 mg). The suspension was degassed by evacuating the flask and refilling with nitrogen (2×). Then the flask was immersed in a 230° C. heating bath for 40 min. After cooling at room temperature the suspension was diluted with ethyl acetate, filtered through a path of Celite and the filtrate was concentrated in vacuo. The residue was purified by column chromatography (SiO₂, ethyl acetate/heptane=1:9) affording 290 mg (68%) of a mixture of CD olefins. GC analysis: 16 (54%); Z isomer (4%); internal olefin (27%); terminal olefin (5%); other impurities (10%).

### (2R,3aR,4S,7aR)-1-E-ethylidene-2-hydroxy-7a-methyl-octahydroinden-4-yl ester (17a) and acetic acid (2S,3aR,4S,7aR)-1-E)-ethylidene-2-hydroxy-7a-methyl-octahydroinden-4-yl ester (17b)

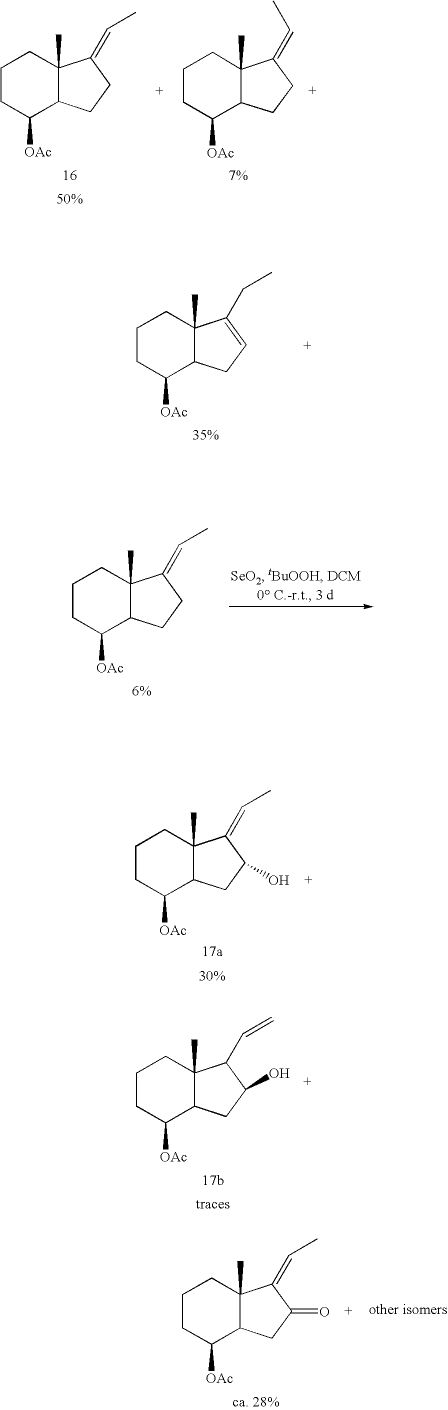

To a suspension of SeO₂ (460 mg, 4.15 mmol) in dichloromethane (30 mL) was added tert.-butylhydroperoxide (9.0 mL, 70 w/w-% solution in water, 65.7 mmol). The suspension was stirred at room temperature for 30 min., cooled at 0° C. and a solution of CD-isomers (9.13 g, 41.1 mmol, contains ca 50% of 16) in dichloromethane (35 mL) was added dropwise within 30 min. The reaction mixture was allowed to reach room temperature overnight and stirring was continued at 30° C. for 2 days. Conversion was checked by GC. The reaction was quenched by addition of water and the aqueous layer was extracted with dichloromethane (3×). The combined organic layers were washed with water (4×), washed with brine, dried (Na₂SO₄), filtered and the filtrate was concentrated in vacuo. The residue was purified by column chromatography (SiO₂, ethyl acetate/heptane=1:3) affording three main fractions: Fraction 1:

Ketone (2.08 g, 42% yield); contaminated with 2 impurities; purity ˜75%; Fraction 2: mixed fraction of alcohol 17a+unwanted isomer (1.32 g); Fraction 3: Alcohol 17a (2.10 g, 42% yield); contaminated with ca. 12% byproduct, but pure enough for further synthesis. Fraction 2 was purified again by column chromatography affording 1.01 g (20% yield) of alcohol 17a contaminated with ca. 20% of an unwanted isomer, but pure enough for further synthesis. *Note: During the oxidation reaction the formation of both isomers 17a and 17b was observed by tlc and GC. After prolonged reaction times the intensity of the lower spot on tlc (mixture of 17b and other isomers) decreased and the formation of ketone was observed. It is important that not only conversion of 16 to alcohol 17a and 17b is complete but also that epimer 17b is completely oxidized to ketone. Epimer 17b can not be separated from unwanted isomers. Retention times on GC: 16 ret. Time=8.06 min; 17 ret. Time=9.10 min; 17b ret. Time=9.30 or 9.34 min; ketone ret. Time=9.60 min. Compound 17a: ¹H NMR: delta 0.94 (s, 3H), 1.30 (m, 1H), 1.40-1.46 (m, 1H), 1.46-1.80 (m, 4H), 1.77 (dd, J=7.2, 1.2 Hz, 3H), 1.80-1.94 (m, 4H), 2.02 (s, 3H), 4.80 (br. s, 1H), 5.23 (m, 1H), 5.47 (qd, J=7.2, 1.2 Hz, 1H). GC-MS: m/e 223 (M−15), 178 (M−60), 163 (M−75). Compound 17b: ¹H NMR: delta 1.24 (s, 3H), 1.38-1.60 (m, 5H), 1.68-1.88 (m, 3H), 1.72 (dd, J=7.2, 1.2 Hz, 3H), 1.99 (ddd, J=11.0, 7.0, 3.7 Hz, 1H), 2.03 (s, 3H), 2.26 (m, 1H), 4.36 (m, 1H), 5.14 (m, 1H), 5.30 (qd, J=7.2, 1.2 Hz, 1H). GC-MS: m/e 223 (M−15), 178 (M−60), 163 (M−75).

### Reduction of Ketone to Alcohol 17b

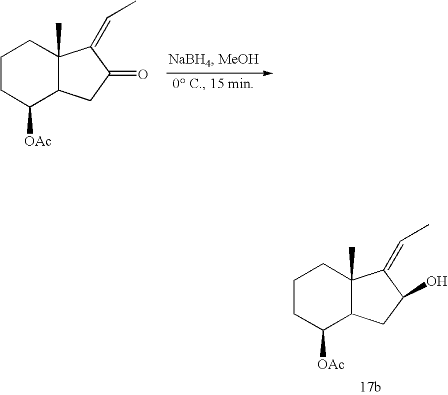

A solution of ketone (2.08 g, contaminated with 2 impurities) in methanol (8 mL) was cooled at 0° C. and sodium borohydride (0.57 g, 15.1 mmol) was added in portions. After stirring at 0° C. for 1 h, tlc showed complete conversion (no UV active compound visible on tlc). The reaction mixture was quenched by addition of sat. aqueous NH₄Cl solution (30 mL). Water was added and the aqueous layer was extracted with ethyl acetate (3×). The combined organic layers were washed with brine, dried (Na₂SO₄), filtered and the filtrate was concentrated in vacuo. The residue was purified by column chromatography (SiO₂, ethyl acetate/heptane=1:3) affording alcohol 17b (1.20 g, 24% over two steps) as a colorless oil.

### Acetic acid (3aR,4S,7aR)-7a-methyl-1-(1-(R)-methyl-3-oxo-propyl)-3a,4,5,6,7,7a-hexahydro-3H-inden-4-yl ester (18)

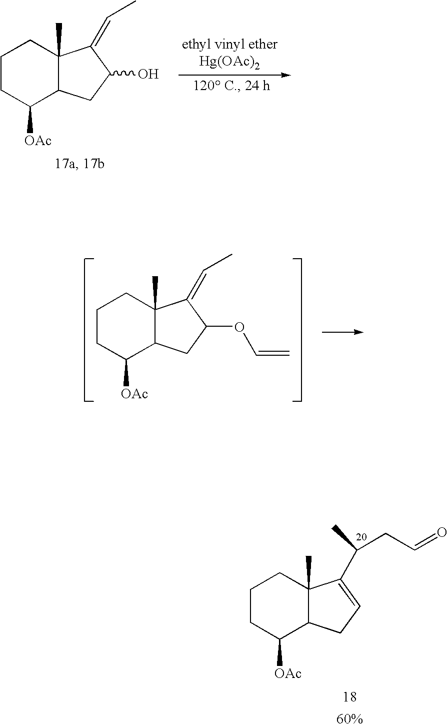

Both alcohols 17a and 17b (4.3 g, 18.1 mmol, purity 90%) were converted to compound 18 in three batches. To a solution of 17a (2.1 g, 8.82 mmol) in ethyl vinyl ether (20 mL) was added Hg(OAc)₂ (2.23 g, 7.00 mmol). The suspension was poured into a pyrex pressure tube, flushed with N₂ and closed tightly. The mixture was stirred at 120° C. for 24 h, cooled at room temperature and filtered. The filtrate was concentrated in vacuo and the residue was combined with the crude product of the two other batches and purified twice* by column chromatography (SiO₂, ethyl acetate/heptane=1:4) affording aldehyde 18 (2.58 g, 60%) as a slightly yellow oil. The product solidified upon storage in the freezer. *a 2^{nd} purification by column chromatography was necessary due to the byproducts present in the starting material.

Alternative Synthesis of Aldehyde 18 (Literature: Okimoto Y et al. J. Am. Chem. Soc., 2002, 124(8), 1590-1591.)

To a solution of epimers 17a and 17b (173 mg, 0.73 mmol) in toluene (2 mL) was added a catalytic amount of [Ir(COD)Cl]₂ (5 mg), Na₂CO₃ (46 mg, 0.44 mmol) and vinyl acetate (0.13 mL, 1.45 mmol). After heating the suspension at 100° C. for 2 h, tlc indicates ca. 20% conversion to intermediate. More vinyl acetate (0.15 mL) was added and stirring at 100° C. was continued for 18 h. According tlc a mixture of intermediate and 18 was formed but conversion of the starting material was still not complete. More vinyl acetate (2 mL) was added and stirring at 100° C. was continued for 24 h. Tlc shows complete conversion of the starting material to a mixture of intermediate and aldehyde 18. The suspension was concentrated in vacuo and the residue was purified by column chromatography (SiO₂, ethyl acetate/heptane=1:9) affording 60 mg of intermediate (31%) and 45 mg of aldehyde 18 (23%). ¹H NMR: delta 1.02 (s, 3H), 1.14 (d, J=7.1 Hz, 3H), 1.36 (M, 1H), 1.47-1.62 (m, 2H), 1.72-1.90 (m, 4H), 2.03 (s, 3H), 2.02-2.14 (m, 2H), 2.33 (ddd, J=16.2, 7.3, 2.6 Hz, 1H), 2.53 (ddd, J=16.2, 5.8, 1.8 Hz, 1H), 2.72 (m, 1H), 5.19 (m, 1H), 5.40 (m, 1H), 9.68 (s, 1H).

### 5(R)-((3aR,4S,7aR)-4-acetoxy-7a-methyl-3a,4,5,6,7,7a-hexahydro-3H-inden-1-yl)-hex-2-E-enoic acid ethyl ester (19)

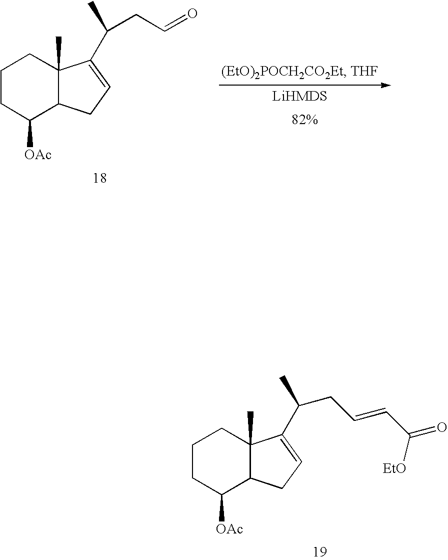

Aldehyde 18 (2.24 g, 8.47 mmol) and triethyl phosphonoacetate (5.74 g, 25.6 mmol, 3 eq.) were dissolved under N₂ atmosphere in THF (40 mL, freshly distilled over Na/benzophenone). The mixture was cooled at −100° C. and a solution of LiHMDS in hexanes (16.8 mL, 1 M solution, 2 eq.) was added dropwise within 20 min. After stirring at −100° C.⇄−78° C. for 70 min. the reaction was quenched by dropwise addition of water (10 mL) and subsequently addition of sat. NH₄Cl solution (10 mL). Water was added and it was extracted with tert. butyl methyl ether (3×). The combined organic layers were washed with water (2×), brine (1×), dried (Na₂SO₄), filtered and the filtrate was concentrated in vacuo. The residue was purified by column chromatography (SiO₂, ethyl acetate/heptane=1:10) affording ester E-19 (2.15 g, 76%) as a colorless oil; purity according NMR: >95% (no Z-isomer detected). ¹H NMR: delta 0.99 (s, 3H), 1.06 (d, J=7.2 Hz, 3H), 1.27 (t, J=7.1 Hz, 3H), 1.36 (td, J=13.3, 4.0 Hz, 1H), 1.46-1.62 (m, 2H), 1.72-1.90 (m, 4H), 1.96-2.17 (m, 3H), 2.03 (s, 3H), 2.22-2.39 (m, 2H), 4.17 (q, J=7.2 Hz, 2H), 5.20 (br. s, 1H), 5.37 (br. s, 1H), 5.78 (dm, J=15.4 Hz, 1H), 6.88 (dt, J=15.4, 7.3 Hz, 1H). HPLC: purity>99% (218 nm). HPLC-MS: m/e 357 (M+23), 275 (M−59).

### (3aR,4S,7aR)-1-((S,E)-5-ethyl-5-hydroxy-1-methyl-hept-3-enyl)-7a-methyl-3a,4,5,6,7,7a-hexahydro-3H-inden-4-ol (20)

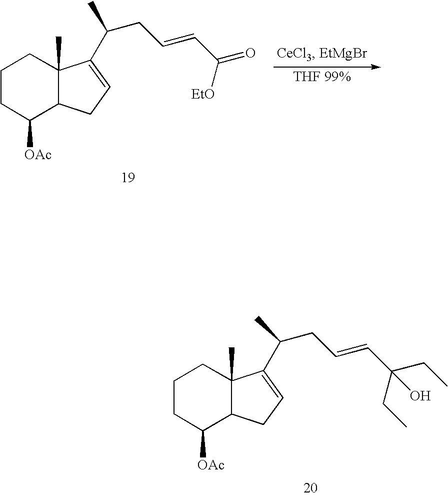

CeCl₃×7H₂O (29.1 g) was dried in vacuo (10⁻³ mbar) in a three-necked flask at 160° C. for 6 h affording anhydrous CeCl₃ (18.7 g, 76.0 mmol, 12 eq.). After cooling at room temperature the flask was purged with nitrogen. THF (200 mL, freshly distilled over Na/benzophenone) was added and the mixture was stirred at room temperature for 18 h. Subsequently the suspension was cooled at 0° C. and a solution of EtMgBr in THF (75 mL, 1 M solution) was added dropwise within 20 min. After stirring the light brown suspension at 0° C. for 2 h a solution of ester E-19 (2.15 g, 6.42 mmol) in THF (30 mL, freshly distilled over Na/benzophenone) was added dropwise within 10 min. After stirring at 0° C. for 30 min. tlc showed complete conversion and the reaction was quenched by addition of water (60 mL). More water was added and the mixture was extracted with 50% ethyl acetate in heptane (3×). The combined organic layers were washed with sat. NaHCO₃ solution (2×), brine (1×), dried (Na₂SO₄), filtered and the filtrate was concentrated in vacuo affording a slightly yellow oil. The crude product (2.4 g) was combined with a 2^{nd} batch (600 mg crude 20 obtained from 550 mg 19). Purification by column chromatography (SiO₂, ethyl acetate/heptane=1:3) afforded 20 (2.45 g, 99%) as a colorless oil. ¹H NMR: delta 0.84 (t, J=7.3 Hz, 6H), 1.04 (d, J=7.2 Hz, 3H), 1.05 (s, 3H), 1.23-1.60 (m, 9H), 1.67-2.02 (m, 6H), 2.12-2.32 (m, 3H), 4.17 (m, 1H), 5.33 (m, 1H), 5.35 (dm, J=15.4 Hz, 1H), 5.51 (ddd, J=15.4, 7.4, 6.5 Hz, 1H). HPLC: purity=98% (212 nm). HPLC-MS: m/e 330 (M+24), 289 (M−17), 271 (M−35).

### (3aR,4S,7aR)-1-((S,E)-5-ethyl-5-hydroxy-1-methyl-hept-3-enyl)-7a-methyl-3a,4,5,6,7,7a-hexahydro-3H-inden-4-one (5)

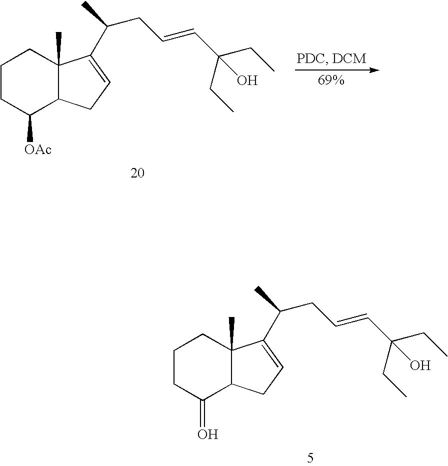

A solution of diol 20 (465 mg, 1.52 mmol) in dichloromethane (30 mL) was cooled in an ice-bath and treated portion-wise with pyridinium dichromate (1.28 g, 3.40 mmol, 2.2 eq.). The reaction mixture was stirred at 0° C. for 6 h and at room temperature for 18 h. The reaction mixture was filtered through a path of Celite. The filtercake was washed with dichloromethane and the combined filtrates were concentrated in vacuo. The residue was purified by column chromatography (SiO₂, 25% ethyl acetate in heptane) affording ketone 5 (320 mg, 69%) as a colorless oil. ¹H NMR: delta 0.82 (s, 3H), 0.85 (br. t, J=7.2 Hz, 6H), 1.05 (d, J=6.9 Hz, 3H), 1.34 (br. s, 1H), 1.52 (br. q, J=6.9 Hz, 4H), 1.65 (td, J=12.1, 5.6 Hz, 1H), 1.84-1.93 (m, 1H), 1.93-2.16 (m, 4H), 2.16-2.33 (m, 4H), 2.42 (ddt, J=15.4, 10.4, 1.6 Hz, 1H), 2.82 (dd, J=10.4, 6.0 Hz, 1H), 5.30 (m, 1H), 5.38 (dm, J=15.6 Hz, 1H), 5.54 (ddd, J=15.6, 7.1, 6.0 Hz, 1H).

### Coupling and Synthesis of (1)

### 1-(5-Ethyl-1-methyl-5-trimethylsilanyloxy-hept-3-enyl)-7a-methyl-3,3a,5,6,7,7a-hexahydro-inden-4-one (22)

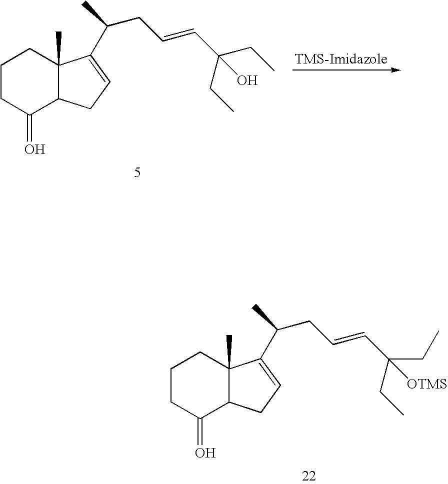

To a solution of compound 5 (320 mg, 1.05 mmol) in dichloromethane (20 mL) was added 1-(trimethylsilyl)imidazole (0.2 mL, 1.34 mmol). The reaction mixture was stirred at room temperature for 4 d. Reaction control (tic) showed complete conversion. The mixture was concentrated in vacuo and the residue was purified by column chromatography (SiO₂, 10% ethyl acetate in heptane) affording compound 22 (377 mg, 95%) as a colorless oil.

### 1 alpha-Fluoro-25-hydroxy-16-23E-diene-26,27-bishomo-20-epi-cholecalciferol (1)

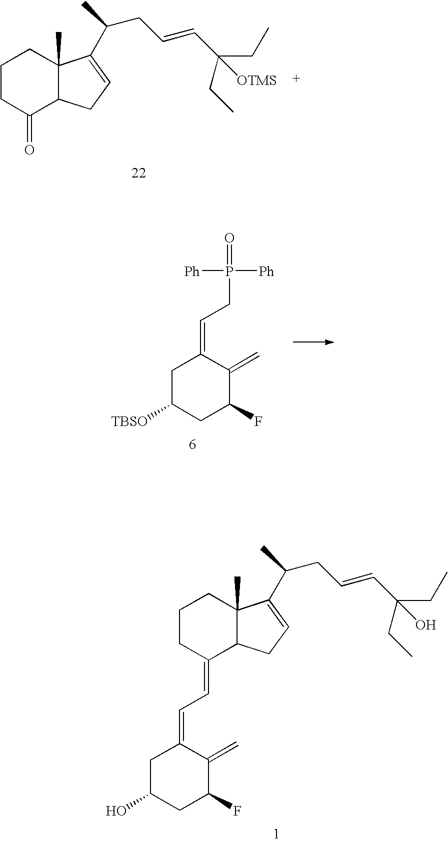

To a stirred solution of 240 mg (0.51 mmole) of 6 in 5 ml of anhydrous tetrahydrofuran at −78° C. was added 0.319 ml (0.51 mmole) of 1.6M n-butyllithium in hexane, dropwise under argon. After stirring for 5 min, to thus obtained red solution was added a solution of 103 mg (0.273 mmole) of 22 in 4 ml of anhydrous tetrahydrofuran, dropwise over a 10 min period. The reaction mixture was stirred at −78° C. for 2 hrs, then placed in freezer (−20° C.) for one hour, quenched by addition of 10 ml of a 1:1 mixture of 2N Rochelle salt and 2N potassium bicarbonate and warmed up to room temperature. After dilution with additional 25 ml of the same salts mixture, it was extracted with 3×90 ml of ethyl acetate. The combined organic layers were washed three times with water and brine, dried over sodium sulfate and evaporated to dryness. The residue was purified by FLASH chromatography on a 30 mm×7″ silica gel column with hexane-ethyl acetate (1:4), to give 145 mg of disilylated title compound. To a solution of 145 mg of disilyl intermediate in 3 ml anhydrous tetrahydrofuran was added 1.7 ml (1.7 mmole) of 1M tetrabutyl-ammonium fluoride in tetrahydrofuran under argon. The reaction mixture was stirred at room temperature for 18 hrs, and then quenched by addition of 10 ml water and stirring for 15 min. It was diluted with 20 ml of water and brine and extracted with 3×80 ml ethyl acetate. The organic layers were washed four times with water and brine, dried over sodium sulfate, and evaporated to dryness. The crude product was purified by FLASH chromatography on a 30 mm×5″ silica gel column with hexane-ethyl acetate (3:2), and by HPLC on a YMC 50 mm×50 cm silica gel column with hexane-ethyl acetate (1:1). It gave 90 mg (74%) of the title compound, crystallization from methyl acetate-hexane.

### Alternate Coupling and Synthesis of 1

### 1-alpha-Fluoro-25-hydroxy-16-23E-diene-26,27-bishomo-20-epi-cholecalciferol (1)

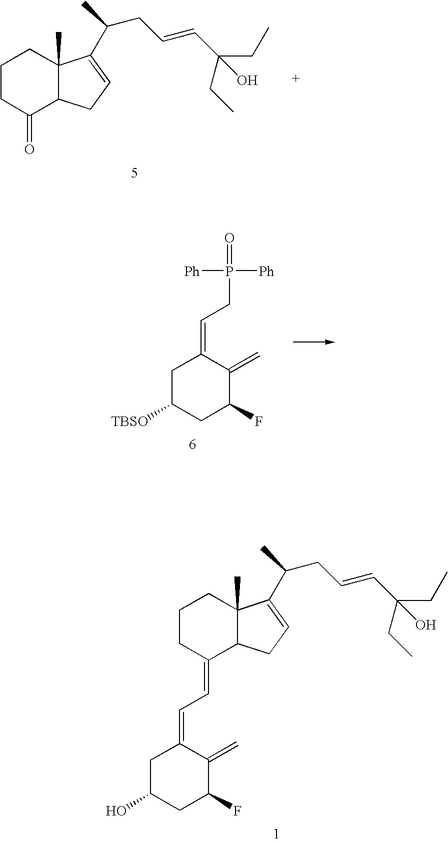

A solution of 6 (278 mg, 0.59 mmol, 3.6 eq.) in THF (10 mL, distilled over Na-benzophenone) was cooled at −75° C. and n-BuLi (0.23 mL, 2.5 M solution in hexanes, 0.57 mmol) was added dropwise. The red solution was stirred for 20 min. during which the temperature was allowed to rise to −50° C. A solution of 5 (50 mg, 0.164 mmol) in THF (2 mL, distilled over Na-benzophenone) was added dropwise at −50° C. within 5 min. Stirring was continued for 2 h during which the temperature was allowed to rise to −10° C. Tlc showed ca. 20% conversion. To the yellow solution was added dropwise TBAF (1.8 mL, 1 M solution in THF, containing ca. 5% water) upon which the solution turned red-brown. The reaction mixture was allowed to reach room temperature overnight. The reaction mixture was quenched by addition of an ice-cold aqueous 1 M KHCO₃ solution (3 g in 30 mL of water) and the mixture was extracted with ethyl acetate (2×40 mL). The combined organic layers were washed with water and brine, dried (Na₂SO₄), filtered and the filtrate was concentrated in vacuo at 30° C. The residue was purified by column chromatography (SiO₂, 25% ethyl acetate in heptane) affording 1 (13 mg, 18%) as a white foam.

### Biological Example 1

### Materials and Methods for the Treatment of Prostate Cancer

Materials: 1-alpha-fluoro-25-hydroxy-16,23E-diene-26,27-bishomo-20-epi-cholecalciferol (Compound 1) was provided by BioXell (Milan, Italy). Anti-KGFR polyclonal antibody was purchased from Santa Cruz Biotechnology, Inc. (Santa Cruz, Calif., USA). Antiphosphotyrosine PY20 antibody and [γ-³²P]ATP were obtained from ICN (Costa Mesa, Calif., USA). Keratinocyte growth factor (KGF) were obtained from Prepro Tech EC (London, England). LY294002 was from Calbiochem (California, USA). Phosphoinositids were from AVANTI POLAR-Lipids, Inc. (Alabaster, Ala., USA). Protein A and Protein G-Sepharose were obtained from Amersham Pharmacia Biotech Italia (Cologno Monzese, Italy). Matrigel was from Becton Dickinson (Franklin Lakes, N.J., USA). Protein measurement Coomassie kit was purchased from Bio-Rad Laboratories, Inc. (Hercules, Calif., USA). Annexin-V-Fluos staining Kit was obtained from Roche Molecular Biochemicals (Milan, Italy). DMEM, antibiotics and other not specified reagents were purchased from SIGMA Chemical Co (St. Louis, Mo., USA).

Cell culture: Androgen independent human cell lines, DU145 and PC3, were obtained from American Tissue Culture Collection (Bethesda, Md., USA) and maintained respectively in DMEM and HAM-F12 Coon supplemented with 10% FBS, penicillin (100 Ul/ml), streptomycin (10 mg/ml) and glutamine (2 mM).

Cell proliferation assay: All proliferation tests were performed after 24 h of cell starvation in phenol red- and serum-free medium containing 0.1% BSA. After starvation, cells were incubated in the same medium as before, with or without specific stimuli. Thereafter, cells were trypsinized, and each experimental point was derived from counting in the hemocytometer and then averaging at least six different fields for each well as previously reported (Crescioli et al. 2003). Experiments were performed seeding 4×10⁴ cells onto 12-well plates in growth medium and incubated for 48 h with: 1) increasing concentrations of 1-alpha-fluoro-25-hydroxy-16,23E-diene-26,27-bishomo-20-epi-cholecalciferol (1×10⁻¹², 1×10⁻¹¹, 1×10⁻¹⁰, 1×10⁻⁹, 1×10⁻⁸ M) with or without fixed concentration of KGF (10 ng/ml) or fixed concentration of bFGF (10 ng/ml); 2) fixed concentration of LY 294002 (10 nM) with or without KGF (10 ng/ml). In the same experiment each experimental point was repeated in triplicate and experiments were performed at least three times. Cell growth results are expressed as the percentage of growth compared with their relative controls.

Invasion assay: Invasion assays were performed as described previously (Bonaccorsi et al. 2000 and 2004a and 2004b) according to Albini et al. (1987) using the Boyden chambers equipped with 8 um porosity polyvinylpyrrolidone-free polycarbonate filters (VWR International, Milan, Italy). A thin layer of Matrigel solution (50 ug/ml) was overlaid on the upper surface of the filter and allowed to gel by incubating the filters at 37° C. for 30 min. Cell ability to invade the substrate was assessed by using some different stimuli: keratinocyte growth factor, KGF (10 ng/ml), in presence or in absence of the inhibitor, 1-alpha-fluoro-25-hydroxy-16,23E-diene-26,27-bishomo-20-epi-cholecalciferol (1×10⁻⁸ M). These molecules were added to the bottom well of the Boyden chambers. 9.5×10⁴ cells were then added to the top of the chambers and incubated for 24 h at 37° C. Migrated cells were quantitated by counting cells with a Zeiss microscope (Oberkochen, Germany) equipped with brightfield optics (40× magnification). Results are expressed as the percentage of number of migrated cells per high-power field respect to control.

Immunoprecipitation and Western blot analysis. Protein extraction and immunoprecipitation were performed as previously described (Bonaccorsi et al. 1997). Briefly, cells were scraped in PBS supplemented with 1 mM Na₃VO₄, centrifuged and resuspended in lysis buffer (20 mM Tris, pH 7.4, 150 mM NaCl, 0.25% NP-40, 1 mM Na₃VO₄, 1 mM phenylmethyl-sulfonyl fluoride (PMSF)). After protein measurement (Coomassie kit), aliquots of cell lysates containing equal amount of proteins (500 ug) were incubated for 1 hour with 30 ul of Protein A (or Protein G)-Sepharose for preclearing. Precleared lysates were then incubated for 1 hour using 5 ug of specific anti-KGFR antibodies on ice followed by overnight incubation at 4° C. with 30 ul of Protein A (or Protein G)-Sepharose. The immunobeads were washed three times in lysis buffer and then resuspended in 10 ul of 2× Laemmli's reducing sample buffer (62.5 mM Tris pH 6.8, 10% glycerol, 2% SDS, 2.5% pyronin and 200 mM dithiothreitol), boiled at 95° C. for 5 minutes and loaded onto 8% polyacrylamide-bisacrylamide gels. After SDS-PAGE, proteins were transferred to nitrocellulose membrane (Sigma Co., St. Louis, Mo., USA) and incubated with the specific primary antibodies for 2 hours in 1% BM blocking (Roche, Milan, Italy) in TTBS solution (Tris-buffered saline containing 0.1% Tween 20, pH 7.4), washed and incubated with peroxidase-conjugated relative secondary antibodies (1:4000) for 2 hours. After washing, the blots were incubated with enhanced chemiluminescence (BM, Roche, Milan, Italy) detection reagent and exposed to film. After the first blotting, nitrocellulose membranes were stripped at 50° C. for 30 min in stripping buffer (100 mM 2β-mercaptoethanol, 2% sodium dodecyl sulphate, 62.5 mM Tris-HCl pH 6.7) and re-probed with specific primary antibodies to detect different proteins.

Annexin-V binding assay. Annexin-V binding assay was used to detect translocation of membrane phosphatidylserine (PS) from the inner to the outer side of the plasma membrane, since the exposure of PS is considered an early sign of apoptosis (Kagan et al. 2000). The assay was performed by using the “Annexin-V-Fluos staining Kit” (Roche). Before treatment, cells (1×10⁶) were kept in serum-free medium for at least 24 h, then cells were incubated for 8 hours in the presence or absence of Compound 1 (1×10⁻⁸ M), cells (1×10⁶) were washed, trypsinized, centrifuged. After two washed in PBS, cells were resuspended in 100 ul of incubation buffer (supplied by manufacturer), 2 ul of Annexin-V-Fluos labeling reagent (Ann-V-F, Annexin-V conjugated to fluorescein, supplied at the 200× concentration by Roche) and 2 ul of propidium iodide solution (PI, 30 ug/ml in PBS) were added. After incubation (15 minutes in the dark at room temperature) samples were analyzed by flow cytometry. For each experimental set, two cell suspensions were prepared for instrumental setting and data analysis: 1) by omitting both Ann-V-F and PI staining (nonspecific fluorescence sample), and 2) by omitting only the PI staining (sample for compensation, see below). Ann-V-F green fluorescence and PI red fluorescence were revealed by using FL-1 and FL-2 detectors, respectively. Fluorescence compensation was set by acquiring sperm labeled with only Ann-V-F. For each sample 10.000 events were recorded at flow rate of 200/300 cells/s. Debris were gated out by establishing a region around the population of interest, in the Forward Scatter/Side Scatter (FSC/SSC) dot plot. Quadrant setting was established in the FL-1/FL-2 dot plot corresponding to the autofluorescence sample by including more than 99% of total events in the lower left quadrant.

PI3K assay: PI3K activity was evaluated in vitro assay as previously described (Luconi et al. 2004). Briefly, cells were stimulated with KGF (5 min) in the presence or absence of 1-alpha-fluoro-25-hydroxy-16,23E-diene-26,27-bishomo-20-epi-cholecalciferol, scraped in PBS supplemented with 1 mM Na₃VO₄, centrifuged and extracted with lysis buffer (20 mM Tris, pH 7.4, 137 mM NaCl, 1 mM CaCl₂, 1 mM MgCl₂, 1% NP-40, 1 mM Na₃VO₄, 1 mM PMSF). After measurement of proteins, the aliquots of cell extracts containing equivalent amount of proteins (500 ug) were incubated for 1 hour with 50 ul of Protein G-Sepharose for preclearing. Precleared lysates were then incubated with an antiphosphotyrosine PY20 antibody overnight at 4° C. with 50 ul of Protein G-Sepharose as described above. The Sepharose beads were washed two times with lysis buffer and twice with a 10 mM Tris-HCl (pH 7.4) containing 0.1 mM EGTA and 5 mM LiCl. After removal of the last wash, the beads were suspended in kinase buffer (10 mM Tris-HCl, 150 mM NaCl, 5 mM EDTA) containing 20 ug of L-alpha-phosphatidylinositol, 25 mM MgCl₂ and 10 uCi of [gamma-³²P]ATP, and incubated for 20 min at room temperature. The reaction was stopped by the addition of 60 ul of HCl 6M and 160 ul of a mixture of chloroform and methanol (1:1). Lipids were then resolved by thin layer chromatography plates, TLC silica gel 60 (Merck Laborchimica, Florence, Italy), in chloroform, methanol, water and ammonium hydroxide (60:47:11.3:2). Dried TLC sheets were developed by autoradiography. Quantifications of the bands was performed using a Kodak image analysis system.

Statistical analysis: All the data are shown as mean±SEM of the indicated number of experiments. Statistical analysis was performed with ANOVA and Student's T test for unpaired and, when applicable, for paired data. IC50 for dose response curves were calculated using the program ALLFIT.

### Biological Example 2

### Inhibitory Effects of 1-alpha-fluoro-25-hydroxy-16,23E-diene-26,27-bishomo-20-epi-Cholecalciferol on Basal and KGF-Mediated Proliferation of DU145 Cells

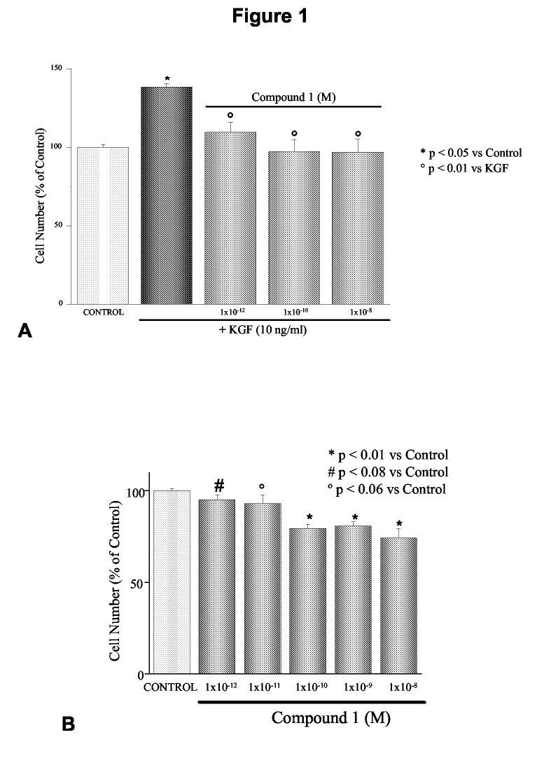

As shown in the inset of FIG. 1, treatment with 1-alpha-fluoro-25-hydroxy-16,23E-diene-26,27-bishomo-20-epi-cholecalciferol (1) inhibited dose dependently DU145 cell proliferation with an IC50 of 22.1±19.1 pM. Similar results were obtained when cell proliferation was assessed using the MTT assay (results not shown). As shown in FIG. 1, KGF stimulates DU145 cell proliferation at the concentration of 10 ng/ml. Treatment with 1-alpha-fluoro-25-hydroxy-16,23E-diene-26,27-bishomo-20-epi-cholecalciferol completely and dose-dependently inhibits proliferation stimulated by the growth factors (FIG. 1). Similar effects were also observed in the androgen-independent cell line PC3 (percentage number of cells: 100±17 control, 121.3±13 KGF [10 ng/ml], 69.9±9.9 KGF+Compound 1 [1×10⁻⁸ M]), although, in line with previous work by our group (Crescioli et al. 2002), responsiveness of PC3 cells to KGF was lower respect to DU145. To evaluate whether the inhibitory effects of 1-alpha-fluoro-25-hydroxy-16,23E-diene-26,27-bishomo-20-epi-cholecalciferol were specific for KGF, its effect was tested on bFGF-stimulated proliferation of DU145 cells. The results demonstrated an inhibitory effect of 1-alpha-fluoro-25-hydroxy-16,23E-diene-26,27-bishomo-20-epi-cholecalciferol also on bFGF-mediated proliferation (percentage number of cells: 100±8.6 control, 138±19.5 bFGF [10 ng/ml], 74.1±9.2 bFGF+Compound 1 [1×10⁻⁸ M]).

Previous studies (Crescioli et al, 2000 and 2002) indicated that vitamin D analogues exert in part their antiproliferative effects by inducing cell apoptosis. To evaluate whether the inhibitory effect of 1-alpha-fluoro-25-hydroxy-16,23E-diene-26,27-bishomo-20-epi-cholecalciferol on DU145 proliferation was due to induction of apoptosis, we evaluated phosphatidylserine exposure (an early sign of cell apoptosis, for review see Kagan et al, 2000) in live cells by Annexin-V binding after 8 hours incubation in the presence of the analogue (1×10⁻⁸ M). We found that 1-alpha-fluoro-25-hydroxy-16,23E-diene-26,27-bishomo-20-epi-cholecalciferol induced a significant increase of Annexin-V binding to the cells (percentage Annexin-V positive live cells: 57±1.7 control, 62±1.2 Compound 1, n=6, p=0.017).

### Biological Example 3

### Effect of 1-alpha-fluoro-25-hydroxy-16,23E-diene-26,27-bishomo-20-epi-cholecalciferol on KGF-Induced Matrigel Invasion of DU145 Cells

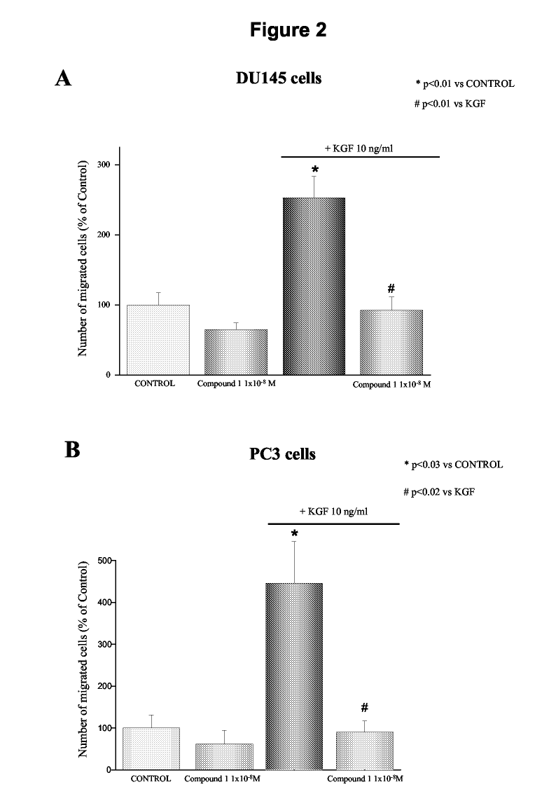

The effect of 1-alpha-fluoro-25-hydroxy-16,23E-diene-26,27-bishomo-20-epi-cholecalciferol (1) on KGF-stimulated Matrigel invasion was investigated. Previous studies investigating the effects of vitamin D analogues on cancer cell invasion and migration, utilized long-term treatment protocols with at least 48 hours cell preincubation before performing the invasion assay (Yudoh et al. 1999; Koli and Keski-Oja 2000; Schwartz et al. 1997). In this study, the effect of 1-alpha-fluoro-25-hydroxy-16,23E-diene-26,27-bishomo-20-epi-cholecalciferol on in vitro invasiveness of DU145 cells was evaluated avoiding pre-incubation of the cells with the analogue, which was added directly to the bottom of Boyden chambers. As shown in FIG. 2 (panel A), the stimulatory effect of KGF on DU145 cell invasion was completely inhibited by the vitamin D analogue at the concentration of 1×10⁻⁸ M. Similar results were observed in the PC3 cell line (FIG. 2, panel B).

### Biological Example 4

### Effects of 1-alpha-fluoro-25-hydroxy-16,23E-diene-26,27-bishomo-20-epi-cholecalciferol on KGF-Induced Signalling Pathways

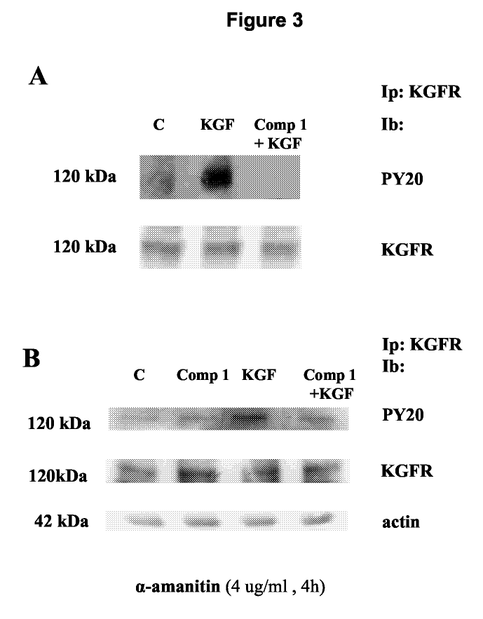

The effects of 1-alpha-fluoro-25-hydroxy-16,23E-diene-26,27-bishomo-20-epi-cholecalciferol (1) on KGF-induced signalling in DU145 cells was investigated. In particular, the effect of 1 on KGFR autotransphosphorylation and on the downstream signalling pathway PI3K/AKT was investigated. Cells were pre-treated for a short time (5 minutes) with 1-alpha-fluoro-25-hydroxy-16,23E-diene-26,27-bishomo-20-epi-cholecalciferol before addition of KGF. For autotransphosphorylation studies, KGF receptor was immunoprecipitated using specific antibody. As shown in FIG. 3A, 1-alpha-fluoro-25-hydroxy-16,23E-diene-26,27-bishomo-20-epi-cholecalciferol abrogated KGF-induced autotransphosphorylation of its receptor. Interestingly, this effect was obtained following a brief (5 minutes) pre-incubation with the analogue, suggesting a rapid, nongenomic effect. To further investigate this possibility, we used the RNA transcription inhibitor alpha-amanitin. As shown in FIG. 3B, the inhibitory effect of 1 was still present when the experiment was conducted following 4 hours incubation with 4 ug/ml alpha-amanitin, strongly indicating absence of transcriptional regulation in the inhibitory effect of 1 on KGFR autophosphorylation.

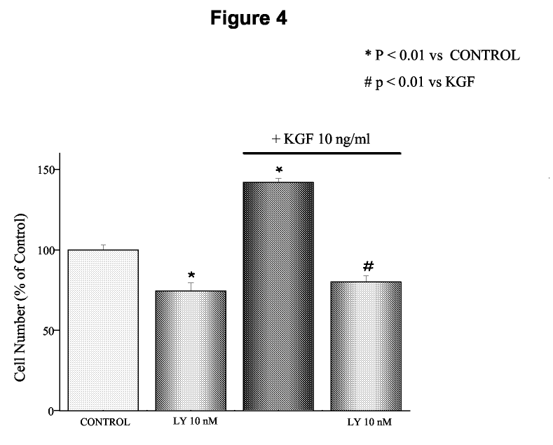

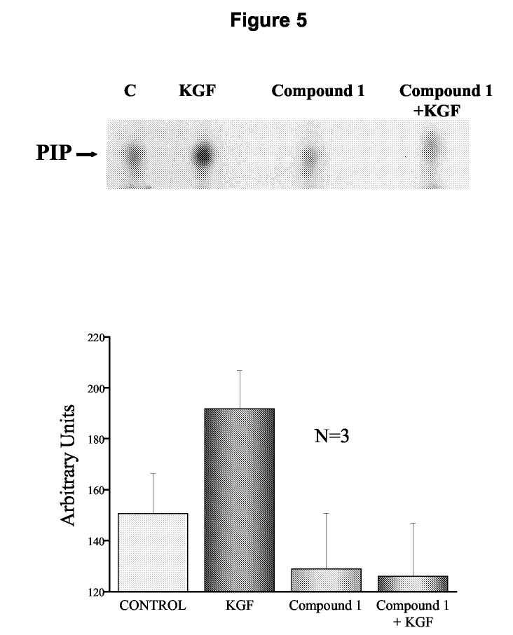

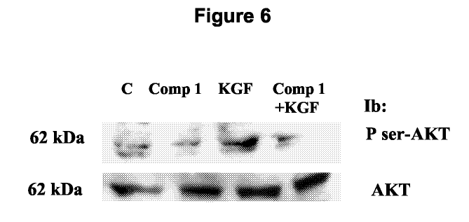

In view of the key role exerted by the PI3K/AKT signalling pathway on invasion and migration of PC3 cells (Bonaccorsi et al. 2004a) as well as on KGF-mediated proliferation of DU145 cells (FIG. 4) where the inhibitory effect of the PI3K inhibitor LY294002 on KGF-induced proliferation of DU145 cells was shown, an evaluation the effect of 1-alpha-fluoro-25-hydroxy-16,23E-diene-26,27-bishomo-20-epi-cholecalciferol on KGF-induced PI3K activation was accomplished. As shown in FIG. 5, 1 inhibited the stimulatory effect of KGF on PI3K activity. Next, the effect of 1 on the main downstream PI3K effector, the serine/threonine kinase AKT (Wyman and Pirola, 1998) was investigated. AKT was activated by serine/threonine phosphorylation following PI3K activation and this phosphorylation was essential for its activity. When AKT was activated, it regulated a variety of cellular functions including cell survival, cell growth, cell differentiation, cell cycle progression and cell metabolism (Paez and Sellers 2003). Thus, an evaluation of serine phosphorylation of AKT was carried out by Western blot analysis, employing a specific anti-serine phosphorylated AKT antibody, following DU145 cell stimulation with KGF in the presence or absence of 1. It was found that 1 inhibited KGF-stimulated AKT serine phosphorylation (FIG. 6) in agreement with the results on receptor autotransphosphorylation (FIG. 3) and PI3K activity (FIG. 5).

Prostate cancer (PC) in advanced stages is a fatal disease because of failure of androgen deprivation therapy and lack of alternative effective therapy. An ideal therapeutic agent for AI-PC should target both proliferation as well as invasive and metastatic properties of the tumor cells, since once progressed to androgen independence, PC is characterized also by higher invasive ability (Chung et al. 2005). The present invention demonstrates that the vitamin D analogue 1-alpha-fluoro-25-hydroxy-16,23E-diene-26,27-bishomo-20-epi-cholecalciferol is able to reduce both proliferation and invasive ability of the AI-PC cell lines, DU145 and PC3, in basal conditions and in response to a main growth factor, namely KGF, implicated in proliferation, progression and invasion of PC (Russell et al. 1998). At least in part, the antiproliferative effect of 1-alpha-fluoro-25-hydroxy-16,23E-diene-26,27-bishomo-20-epi-cholecalciferol is due to induction of apoptosis, as demonstrated by increased surface exposure of PS in live cells after treatment with the analogue. This result is in line with previous data showing induction of apoptosis by vitamin D analogues in several cell types (Crescioli et al. 2000, 2002, 2003 and 2004).

KGF is a physiological paracrine factor for prostate epithelial cells produced by stromal cells under the control of androgen (Planz et al. 1999a), but in the case of PC the paracrine loop is mostly replaced by an autocrine one (Planz et al. 1999b) with enhanced effectiveness on cell proliferation. Thus, the growth factor and its receptor represent an important target for therapeutic strategies in advanced PC. Several receptor tyrosine kinase (RTK) inhibitors have been developed in recent years to specifically block receptor tyrosine kinases such as EGFR, VGFR and FGFR (Noble et al. 2004). Among these, Gefitinib, an inhibitor of EGFR tyrosine kinase, has been shown to effectively block, in vitro, EGFR signalling and EGF mediated proliferation and invasion of PC cell lines (Vicentini et al. 2003; Bonaccorsi et al. 2004b). However, despite clear effectiveness in other solid tumors (Blay et al. 2005), results of a phase II clinical trial for PC with this inhibitor were disappointing (Canil et al. 2005). Lack of effectiveness of Gefitinib has been demonstrated also for renal and bladder cancers (Drucker et al. 2003; Petrylak et al. 2003) although abnormal EGFR expression/signaling has been demonstrated in these malignancies, suggesting tissue selectivity for these agents. It is likely that combination with other therapies is required for the treatment of these malignancies. It is demonstrated here that 1-alpha-fluoro-25-hydroxy-16,23E-diene-26,27-bishomo-20-epi-cholecalciferol, consistent with previous results obtained with vitamin D analogue 1,25-dihydroxy-16ene-23yne vitamin D₃ (Crescioli et al. 2002), is able to inhibit, as RTK inhibitors, KGFR autotransphosphorylation in DU145 cells through a rapid, likely nongenomic, mechanism of action. The demonstration that the downstream PI3K/AKT pathway is inhibited, suggests that 1-alpha-fluoro-25-hydroxy-16,23E-diene-26,27-bishomo-20-epi-cholecalciferol is effective in blocking KGF action. As mentioned above, 1-alpha-fluoro-25-hydroxy-16,23E-diene-26,27-bishomo-20-epi-cholecalciferol is less hypercalcemic compared to calcitriol and other analogues. In addition, 1-alpha-fluoro-25-hydroxy-16,23E-diene-26,27-bishomo-20-epi-cholecalciferol is currently being tested in phase II clinical trials for the treatment of BPH and preliminary results indicate significant reduction of prostate volume compared to placebo adverse effects (Montorsi F, presentation at the EUA, Instanbul, March 2005), strongly indicating that the prostate is a target for this drug.

The instant invention shows that the non hypercalcemic vitamin D analogue, 1-alpha-fluoro-25-hydroxy-16,23E-diene-26,27-bishomo-20-epi-cholecalciferol, is able to block proliferation and invasion in response to KGF in the Al cell line DU145. Together with several evidence in the literature pointing out a differentiating role of calcitriol and its analogues in carcinoma cells (Stewart and Weigel 2004), our data provide a rationale for the development of novel analogues to be employed in the treatment of androgen-independent or advanced PC.

### REFERENCES

Baldi et al. 2003 Endocrinology 144:1653-1655.
Bergman, T. and Postlind, H. (1991) Biochem. J. 276:427-432.
Bikle, D. D. et al. (1986) J. Clin. Endocrinol. Metab. 63:954-959.
Blay J Y, et al. (2005) Bull Cancer 92:E13-18.
Blunt, J. W. et al. (1968) Biochemistry 6:3317-3322.
Bonaccorsi L, et al. (1997) Biochim Biophys Acta 1355: 155-166.
Bonaccorsi L, et al. (2000) Endocrinology 141: 3172-3182.
Bonaccorsi L, et al. (2004a) Int J Cancer 112:78-86.
Bonaccorsi L, et al. (2004b) J Cancer Res Clin Oncol 130:604-614.
Bouillon, R. et al. Endocrine Reviews 16(2):201-204.
Canil C M, et al. (2005) J Clin Oncol 23:455-460.
Chung L W, et al. (2005) J Urol 173:10-20.
Cooke, N.E. and Haddad, J. G. (1989) Endocr. Rev. 10:294-307.
Crescioli C, et al. (2000) J Clin Endocrinol Metab 85:2576-2583.
Crescioli C, et al. (2002) Prostate 50:15-26.
Crescioli C, et al. (2003) Endocrinology 144:3046-3057.
Crescioli C, et al. (2004) Eur J Endocrinol 150:591-603.
Drucker B. et al. (2003) Invest New Drugs 21:341-345.
Fraser, D. R. and Kodicek, E (1970) Nature 288:764-766.
Gray, R. W. and Ghazarian, J. G. (1989) Biochem. J. 259:561-568.
Gronberg H. (2003) Lancet 361:859-864.
Habuchi et al. 2000 Cancer Res 60:305-308.
Haussler, M. R. et al. (1969) Exp. Cell Res. 58:234-242.
Henry, H. L. and Norman, A. W. (1974) J. Biol. Chem. 249:7529-7535.
Hochberg, R. B., (1998) Endocr Rev. 19(3): 331-348.
Holick, M. F. (1971) Proc. Natl. Acad. Sci. USA 68:803-804.
Kagan et al (2000) FEBS Lett 477:1-7.
Koli K, Keski-Oja J (2000) Cell Growth Differ 11:221-229.
Lawson, D. E. M. et al. (1971) Nature 230:228-230.
Luconi et al (2004) J Cell Sci 117: 1235-1246.
Mellanby, E. (1921) Spec. Rep. Ser. Med. Res. Council (GB) SRS 61:4.
Myrtle, J. F. et al. (1970) J. Biol. Chem. 245:1190-1196.
Nagpal et al, 2005 Endocr Rev. 26:662-87.
Noble M E, et al. (2004) Science 303:1800-1805.
Norman, A. W. et al. (1971) Science 173:51-54.
Norman, A. W. et al. (1993) J. Biol. Chem. 268 (27): 20022-20030.
Ohyama, Y. and Okuda, K. (1991) J. Biol. Chem. 266:8690-8695.
Okimoto Y et al. J. Am. Chem. Soc., 2002, 124(8), 1590-1591.
Paez J. Sellers W R (2003) Cancer Treat Res 115:145-167.
Petrylak D, et al. (2003) Proc. Am Soc Clin Oncol 22:403 (abstract 1619).
Pheel and Feldman 2004 J Steroid Biochem Mol Biol 92:307-315.
Pike, J. W. (1991) Annu. Rev. Nutr. 11:189-216.
Planz B. et al. (1999a) Prostate 41:233-242.
Planz B. et al. (1999b) J Urol 161:1329-1336.
Radinov et al. J. Org. Chem. 2001, 66, 6141.
Reichel, H. and Norman, A. W. (1989) Annu. Rev. Med. 40:71-78.
Russell P J, et al. (1998) Clin Chem 44: 705-723.
Schwartz G G, et al. (1997) Cancer Epidemiol Biomarkers Prev 6:727-732.
So, et al. 2005 J Urol 23:1-9.
Stewart L V, Weigel N L (2004) Exp Biol Med (Maywood) 229:277-284.
Sung and Feldman. 2000 Mol Cell Endocrinol 164:133-143.
Trump et al, 2004 J Steroid Biochem Mol Biol 89-90:519-26
Tsai, H. C. and Norman, A. W. (1972) J. Biol. Chem. 248:5967-5975.
Van Baelen, H. et al. (1988) Ann NY Acad. Sci. 538:60-68.
Vicentini C, et al. (2003) J Cancer Res Clin Oncol 129: 165-174.
Wymann M P, Pirola L (1998) Biochim Biophys Acta 1436:127-150.
Wong, R. G. et al. (1972) J. Clin. Invest. 51:1287-1291.
Yudoh K, et al. (1999). J Lab Clin Med 133:120-128.
“Chiral Liquid Chromatography,” W. J. Lough, Ed. Chapman and Hall, New York (1989).
EP808833
U.S. Pat. No. 5,939,408
U.S. Pat. No. 6,255,501
WO 97/11053.

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

1. A method for preventing or treating prostate cancer in a subject, comprising administering to a subject in need thereof a therapeutically effective amount of a vitamin D compound.

2-6. (canceled)

7. The method of claim 1, wherein the vitamin D compound is a compound of formula (I): 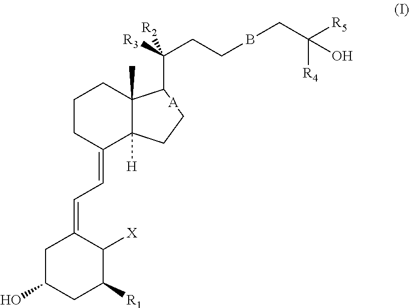
wherein:
X is H₂ or CH₂;
R₁ is hydrogen, hydroxy or fluoro;
R₂ is hydrogen or methyl;
R₃ is hydrogen or methyl, wherein both R₂ and R₃ cannot both be hydrogen;
R₄ is methyl, ethyl or trifluoromethyl;
R₅ is methyl, ethyl or trifluoromethyl;
A is a single or double bond; and
B is a single, E-double, Z-double or triple bond; and pharmaceutically acceptable esters, salts, and prodrugs thereof.

8. The method according to claim 7, wherein A is a double bond.

9. The method according to claim 7, wherein B is an E-double bond.

10. The method according to claim 7, wherein X is CH₂.

11. The method according to claim 7, wherein R₁ is fluoro.

12. The method according to claim 7, wherein R₂ is hydrogen.

13. The method according to claim 7, wherein R₃ is methyl.

14. The method according to claim 7, wherein R₄ and R₅ are each ethyl.

15. The method according to claim 7, wherein A is a double bond, B is an E-double bond, and X is CH₂.

16. The method according to claim 1, wherein the vitamin D compound is 1-alpha-fluoro-25-hydroxy-16,23E-diene-26,27-bishomo-20-epi-cholecalciferol (Compound 1):

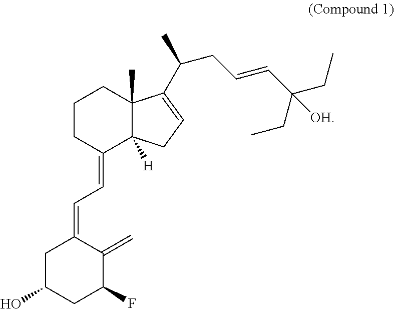

17. The method according to claim 1, further comprising the step of identifying a subject in need of prevention or treatment for prostate cancer.

18. The method according to claim 1, further comprising the step of obtaining the vitamin D compound of formula (I).

19. The method according to claim 1, wherein the subject is a mammal.

20. The method according to claim 19 wherein the subject is a human.

21. The method according to claim 1, wherein the vitamin D compound of formula (I) is formulated in a pharmaceutical composition together with a pharmaceutically acceptable diluent or carrier.

22. The method according to claim 1, wherein the vitamin D compound is administered separately, sequentially or simultaneously in separate or combined pharmaceutical formulations with a further agent for the treatment or prevention of prostate cancer.

23. The method according to claim 22, wherein the further agent is an alpha-adrenergic receptor blocking agent.

24. The method according to claim 22, wherein the alpha-adrenergic receptor blocking agent is selected from terazosin, doxazosin, tamsulosin, silodosin, AIO-8507L and RBx-2258.

25. The method according to claim 22, wherein the further agent is a 5 alpha-reductase inhibitor.

26. The method according to claim 25, wherein the 5 alpha-reductase inhibitor is selected from finasteride and dutasteride.

27. The method according to claim 1, wherein the vitamin D compound is provided in unit dose form.

28. The method according to claim 27, wherein the unit dose form of the vitamin D compound is 50 to 150 μg.

29. The method according to claim 1, for the prevention or treatment of prostate cancer without anti-androgenic prostatic and extra-prostatic adverse effects.

30. The method according to claim 1 in the prevention or treatment of androgen independent prostate cancer.

31. The method according to claim 1, for the prevention of prostate cancer.

32. The method according to claim 1, for the treatment of prostate cancer.

33. A pharmaceutical composition comprising a therapeutically effective amount for the treatment or prevention of prostate cancer of a vitamin D compound, and a pharmaceutically acceptable carrier.

34. The pharmaceutical composition of claim 33, comprising a further agent for the treatment or prevention of prostate cancer.

35. The pharmaceutical composition of claim 33, wherein the vitamin D compound is a compound of formula (I): 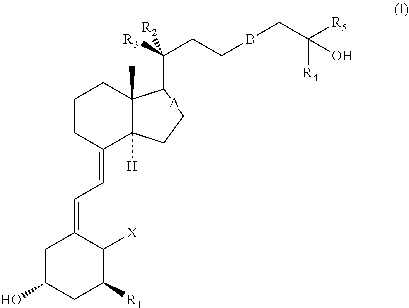
wherein:
X is H₂ or CH₂;
R₁ is hydrogen, hydroxy or fluoro;
R₂ is hydrogen or methyl;
R₃ is hydrogen or methyl, wherein both R₂ and R₃ cannot both be hydrogen;
R₄ is methyl, ethyl or trifluoromethyl;
R₅ is methyl, ethyl or trifluoromethyl;
A is a single or double bond; and
B is a single, E-double, Z-double or triple bond; and pharmaceutically acceptable esters, salts, and prodrugs thereof.

36. A kit comprising a vitamin D compound, packaged together with instructions directing administration of said compound to a subject in need of treatment or prevention of prostate cancer.

37. The kit of claim 36, wherein the vitamin D compound is a compound of formula (I): 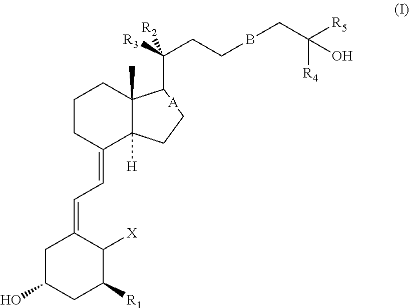
wherein:
X is H₂ or CH₂;
R₁ is hydrogen, hydroxy or fluoro;
R₂ is hydrogen or methyl;
R₃ is hydrogen or methyl, wherein both R₂ and R₃ cannot both be hydrogen;
R₄ is methyl, ethyl or trifluoromethyl;
R₅ is methyl, ethyl or trifluoromethyl;
A is a single or double bond; and
B is a single, E-double, Z-double or triple bond; and pharmaceutically acceptable esters, salts, and prodrugs thereof.

38. A pharmaceutical combination comprising a vitamin D compound and a further agent for the treatment or prevention of prostate cancer.
